(19) United States
(12) Patent Application Publication
(10) Pub. No.: US 2009/0149397 A1
(43) Pub. Date: Jun. 11, 2009

(54) TREATMENT OF CANCER WITH COMBINATIONS OF TOPOISOMERASE INHIBITORS AND PARP INHIBITORS

(75) Inventors: Valeria Ossovskaya, San Francisco, CA (US); Charles Bradley, Half Moon Bay, CA (US); Barry Sherman, Hillsborough, CA (US)

Correspondence Address: WILSON SONSINI GOODRICH & ROSATI, 650 PAGE MILL ROAD, PALO ALTO, CA, 94304-1050, US

(73) Assignee: BiPar Sciences

(21) Appl. No.: 12/329,503

(22) Filed: Dec. 5, 2008

Related U.S. Application Data
(60) Provisional application No. 61/012,364, filed on Dec. 7, 2007.

(51) Int. Cl. A61K 31/166; A61P 35/00; A61K 31/7028; A61K 31/4545; A61K 31/4375; A61K 31/37; A61K 31/496; A61K 31/437; A61K 31/195

(52) U.S. Cl. 514/25; 514/562; 514/618; 514/619; 514/622; 514/283; 514/253.02; 514/279; 514/457

(57) ABSTRACT

In one aspect, the present invention provides a composition and a kit comprising a combination of topoisomerase inhibitor and PARP inhibitor for treatment of cancer. In another aspect, the invention provides a method of treating cancer comprising administering to a subject a combination of topoisomerase inhibitor and PARP inhibitor. In particular, the invention provides compositions and methods for treating cancer in a subject by inhibiting a poly-ADP-ribose polymerase and a topoisomerase, as well as providing formulations and modes of administering such compositions.

### CROSS REFERENCE

This application claims the benefit of U.S. Provisional Application No. 61/012,364, entitled “Treatment of Cancer with Combinations of Topoisomerase Inhibitors and PARP Inhibitors” filed Dec. 7, 2007 (Attorney Docket No. 28825-747.101), which is incorporated herein in its entirety by reference.

### BACKGROUND OF THE INVENTION

Cancer is a serious public health threat. Malignant cancerous growths, due to their unique characteristics, pose serious challenges for modern medicine. These characteristics include uncontrollable cell proliferation resulting in unregulated growth of malignant tissue, an ability to invade local and even remote tissues, lack of control of cellular differentiation and often the lack of effective therapy and prevention.

Cancer can develop in any tissue of any organ at any age. The etiology of cancer has not been fully elucidated; but mechanisms such as genetic susceptibility, chromosome breakage disorders, viruses, environmental factors and immunologic disorders have all been linked to a malignant cell growth and transformation. Cancer encompasses a large category of medical conditions, affecting millions of individuals worldwide. All cancer types begin with the out-of-control growth of abnormal cells.

There are many types of cancer, including, lung, bladder, prostate, pancreatic, cervical, brain, gastric, colorectal and melanoma. Currently, some of the main treatments available are surgery, radiation therapy, and chemotherapy. Surgery is often a drastic measure and can have serious consequences. For example, some treatments for cervical cancer, bladder cancer, prostate cancer or testicular cancer may cause infertility and/or sexual dysfunction. Surgical procedures to treat pancreatic cancer may result in partial or total removal of the pancreas and can carry significant risks to the patient. Some surgical procedures for prostate cancer carry the risk of urinary incontinence and impotence. The procedures for lung cancer patients often have significant post-operative pain as the ribs must be cut through to access and remove the cancerous lung tissue. In addition, patients who have both lung cancer and another lung disease, such as emphysema or chronic bronchitis, typically experience an increase in their shortness of breath following the surgery.

Radiation therapy has the advantage of killing cancer cells but it also damages non-cancerous tissue at the same time. Chemotherapy involves the administration of various anti-cancer drugs to a patient but often is accompanied by adverse side effects.

Worldwide, more than 10 million people are diagnosed with cancer every year and it is estimated that this number will grow to 15 million new cases every year by 2020. Cancer causes six million deaths every year or 12% of the deaths worldwide. There remains a need for methods that can treat cancer. These methods can provide the basis for pharmaceutical compositions useful in the prevention and treatment of cancer in humans and other mammals.

### SUMMARY OF THE INVENTION

In one aspect, the present invention provides a method of treating a cancer, comprising administering to a patient an effective amount of a combination of a topoisomerase inhibitor and a PARP inhibitor of formula (Ia)

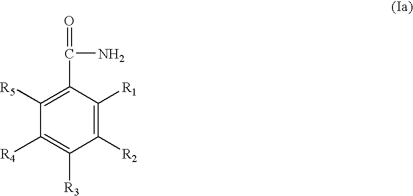

wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof; and wherein the cancer is not breast cancer, uterine cancer, or ovarian cancer.

In some embodiments of the method, the PARP inhibitor is of formula:

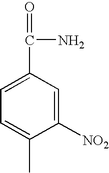

In some embodiments of the method, the PARP inhibitor is a metabolite of 4-iodo-3-nitrobenzamide selected from the group consisting of:

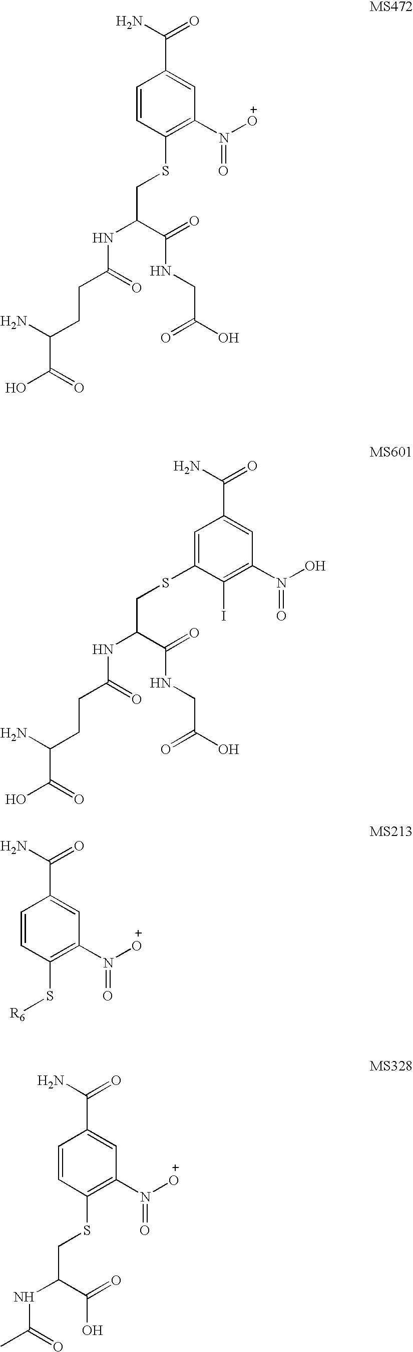

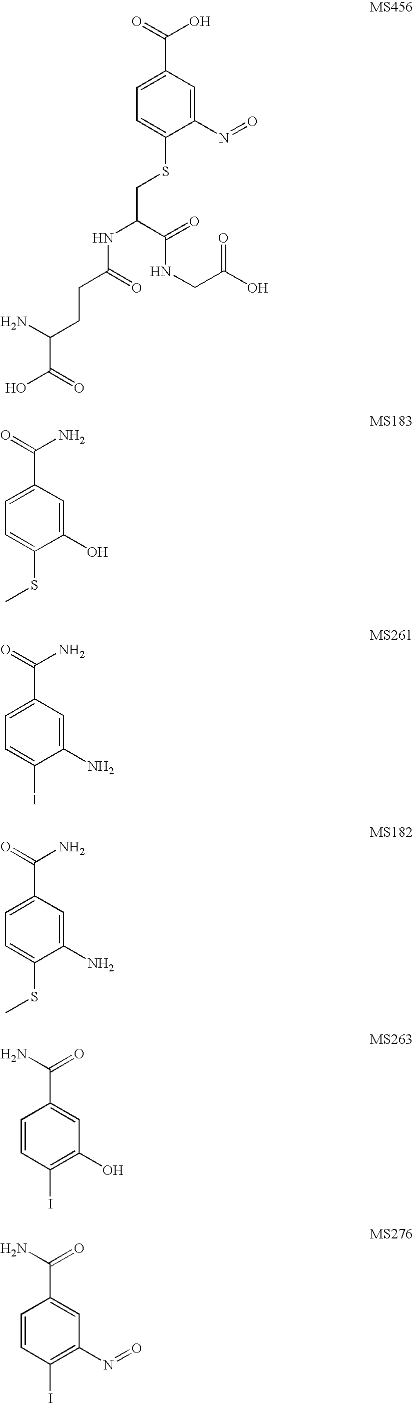

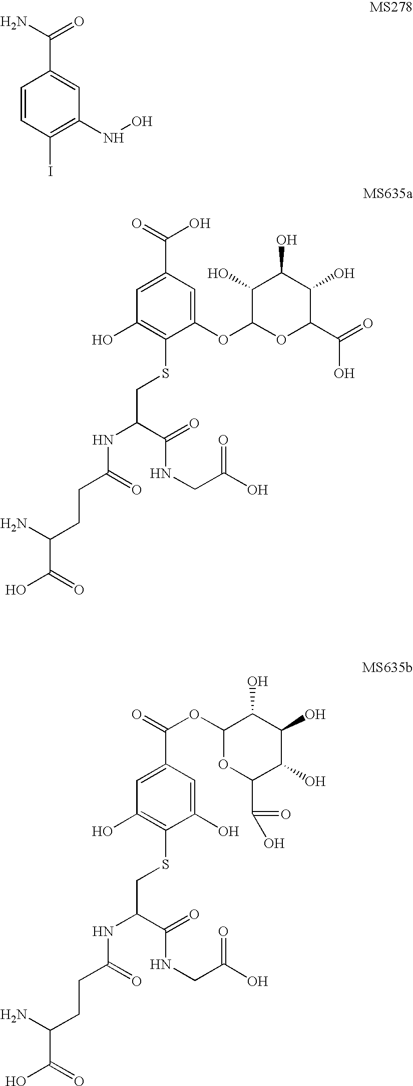

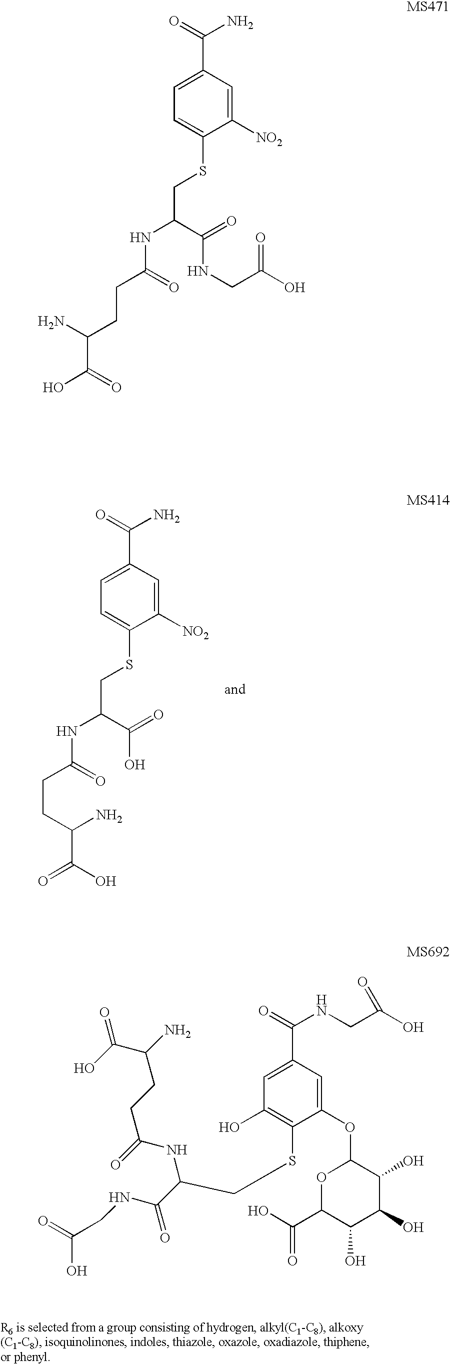

In some embodiments of the method, the topoisomerase inhibitor is topotecan, irinotecan, lurtotecan, exatecan or a pharmaceutically acceptable salt or metabolite thereof. In some embodiments, the topoisomerase inhibitor is topotecan or a pharmaceutically acceptable salt or metabolite thereof. In some embodiments, the cancer is selected from adrenal cortical cancer, anal cancer, aplastic anemia, bile duct cancer, bladder cancer, bone cancer, bone metastasis, CNS tumors, peripheral CNS cancer, Castleman's Disease, cervical cancer, childhood Non-Hodgkin's lymphoma, colon and rectum cancer, esophagus cancer, Ewing's family of tumors, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, vaginal cancer, vulvar cancer, Waldenstrom's macroglobulinemia and cancers of viral origin. In some embodiments, the cancer is selected from the group consisting of leukemia, prostate cancer, transitional cell carcinoma of the bladder, pancreatic cancer, colorectal cancer, cervical cancer, and lung cancer.

In some embodiments, the method of the present invention further comprises administering an effective amount of a benzopyrone compound of formula (II):

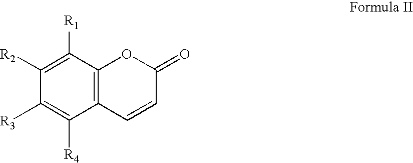

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

In some embodiments of the method, at least one therapeutic effect is obtained, said at least one therapeutic effect being reduction in size of a tumor, reduction in metastasis, complete remission, partial remission, pathologic complete response, or stable disease. In some embodiments, an improvement of clinical benefit rate (CBR=CR+PR+SD≧6 months) is obtained as compared to treatment with the topoisomerase inhibitor but without the PARP inhibitor. In some embodiments, the improvement of clinical benefit rate is at least about 60%. In some embodiments, the method further comprises surgery, radiation therapy, chemotherapy, gene therapy, DNA therapy, adjuvant therapy, neoadjuvant therapy, viral therapy, RNA therapy, immunotherapy, nanotherapy or a combination thereof. In some embodiments, the topoisomerase inhibitor is administered as an intravenous infusion. In some embodiments, 4-iodo-3-nitrobenzamide or its metabolite is administered orally or as a parenteral injection or infusion, or inhalation. In some embodiments, the PARP inhibitor is administered prior to, or concurrently with, or subsequent to the administration of the topoisomerase inhibitor. In some embodiments, the PARP inhibitor and the topoisomerase inhibitor are administered in the same formulation. In some embodiments, the PARP inhibitor and the topoisomerase inhibitor are administered in different formulations.

In another aspect, the present invention provides a composition for administration to a patient for the treatment of cancer, the composition comprising an effective amount of a combination of a topoisomerase inhibitor and a PARP inhibitor of formula (Ia):

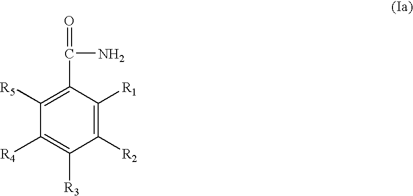

wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs or prodrugs thereof; and wherein the cancer is not breast cancer, uterine cancer, or ovarian cancer.

In some embodiments of the composition, the PARP inhibitor is of formula:

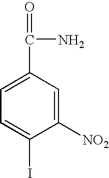

In some embodiments of the composition, the PARP inhibitor is a metabolite of 4-iodo-3-nitrobenzamide selected from the group consisting of:

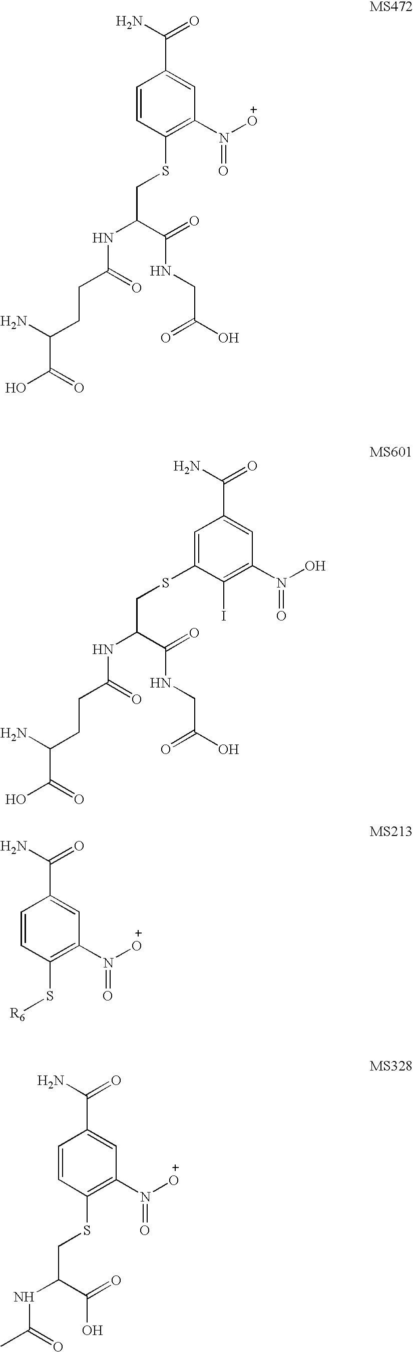

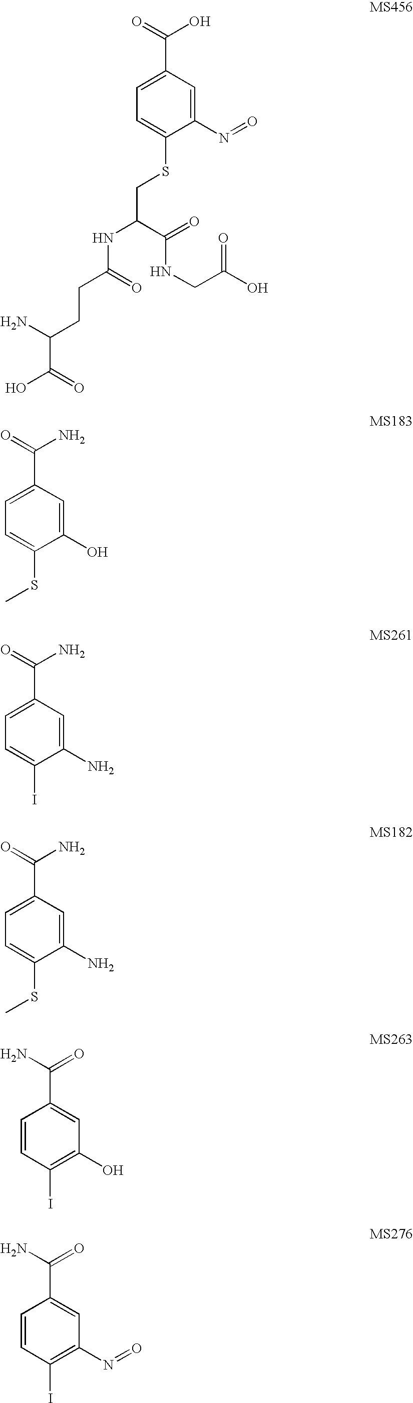

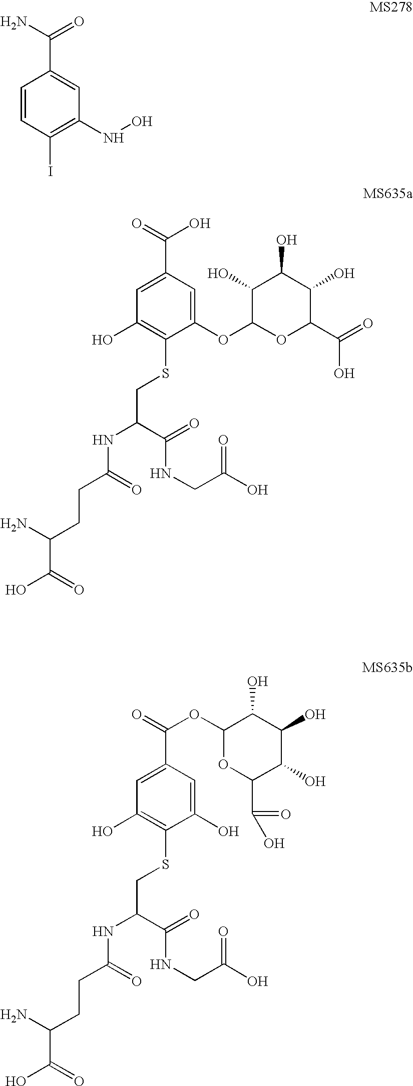

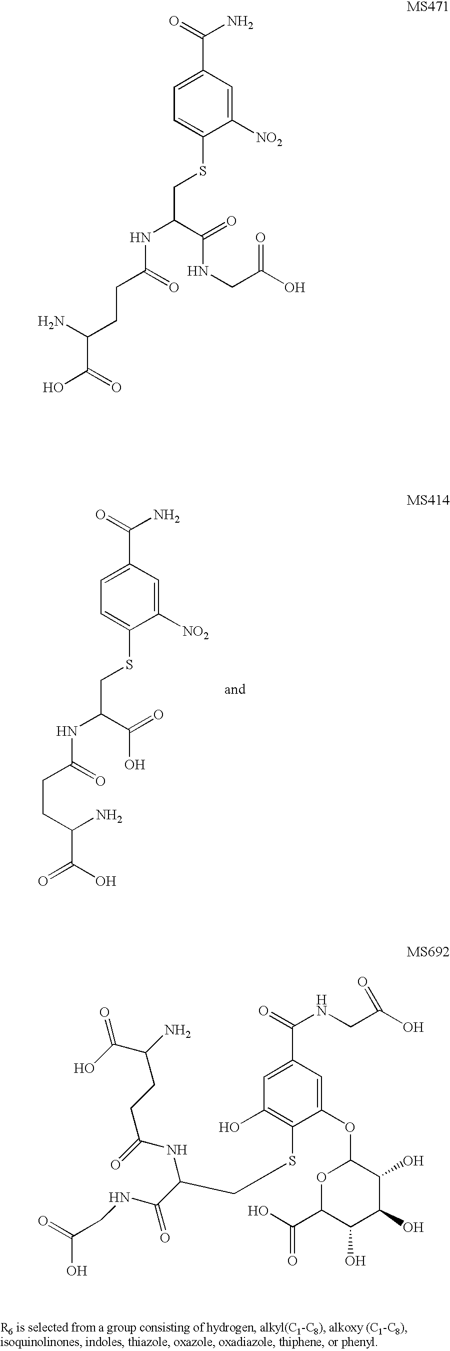

In some embodiments of the composition, the topoisomerase inhibitor is topotecan, irinotecan, lurtotecan, exatecan or a pharmaceutically acceptable salt or metabolite thereof. In some embodiments, the topoisomerase inhibitor is topotecan or a pharmaceutically acceptable salt or metabolite thereof. In some embodiments, the cancer is selected from the group consisting of leukemia, prostate cancer, transitional cell carcinoma of the bladder, pancreatic cancer, colorectal cancer, cervical cancer, and lung cancer. In some embodiments, the composition further comprises an effective amount of a benzopyrone compound of formula (II):

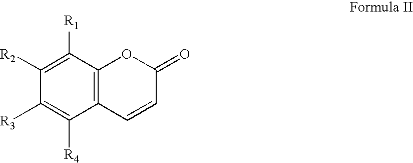

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

In some embodiments, the composition is administered in unit dosage form. In some embodiments, the unit dosage form is adapted for oral or parenteral administration. In some embodiments, upon administration of the composition, at least one therapeutic effect is obtained, said at least one therapeutic effect being reduction in size of a tumor, reduction in metastasis, complete remission, partial remission, pathologic complete response, or stable disease. In some embodiments, upon administration of the composition, an improvement of clinical benefit rate (CBR=CR+PR+SD≧6 months) is obtained as compared to treatment with the topoisomerase inhibitor but without the PARP inhibitor. In some embodiments, the improvement of clinical benefit rate is at least about 60%. In some embodiments, the composition is administered in combination with surgery, radiation therapy, chemotherapy, gene therapy, DNA therapy, adjuvant therapy, neoadjuvant therapy, viral therapy, RNA therapy, immunotherapy, nanotherapy or a combination thereof.

In yet another aspect, the present invention provides a kit for treatment of cancer, comprising: (a) a PARP inhibitor of the formula (Ia):

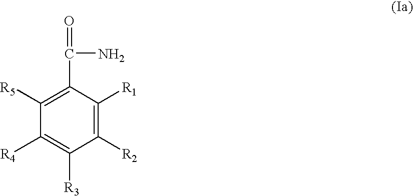

wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs or prodrugs thereof; and (b) a topoisomerase inhibitor; wherein the cancer is not breast cancer, uterine cancer, or ovarian cancer.

In some embodiments of the kit, the PARP inhibitor is of formula:

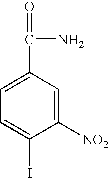

In some embodiments of the kit, the PARP inhibitor is a metabolite of 4-iodo-3-nitrobenzamide selected from the group consisting of:

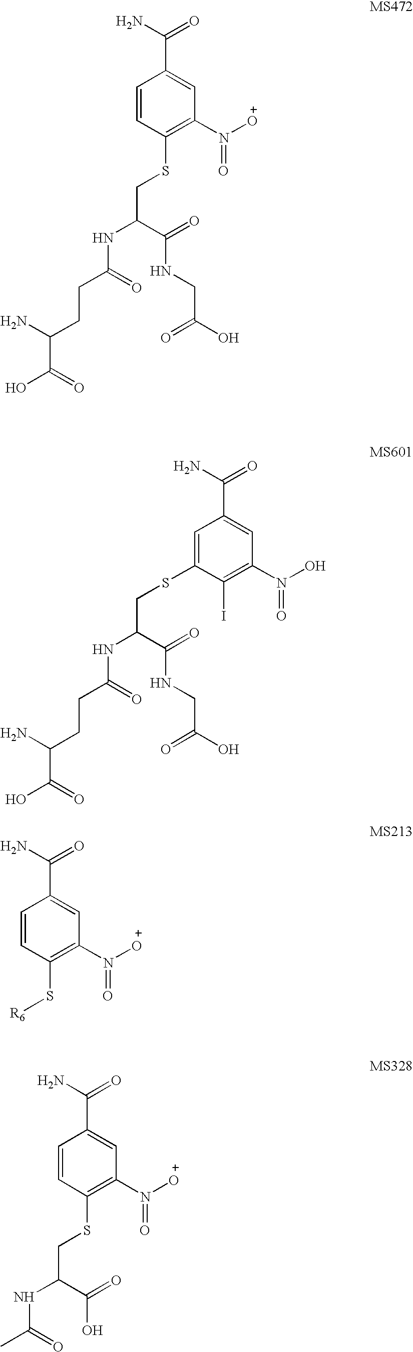

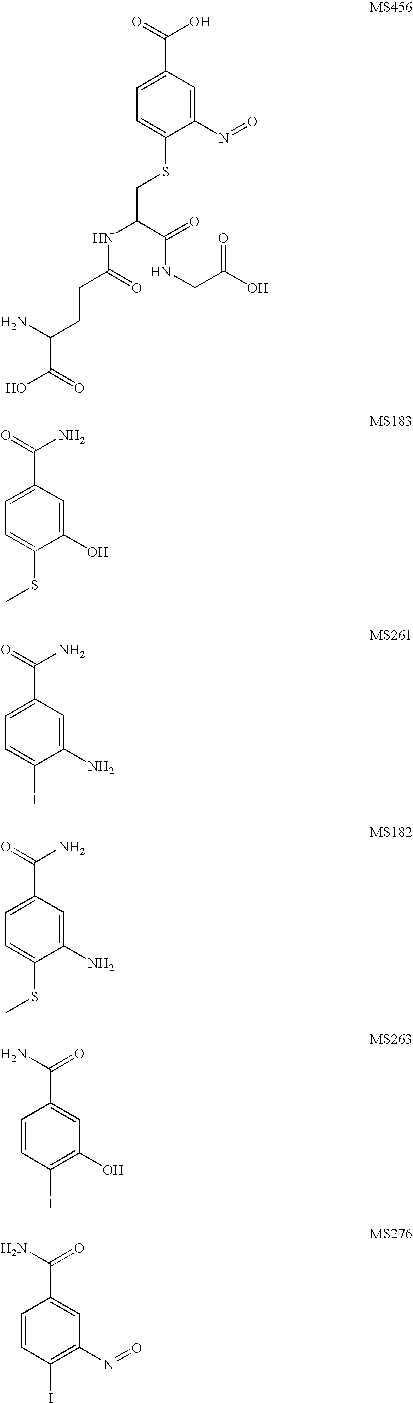

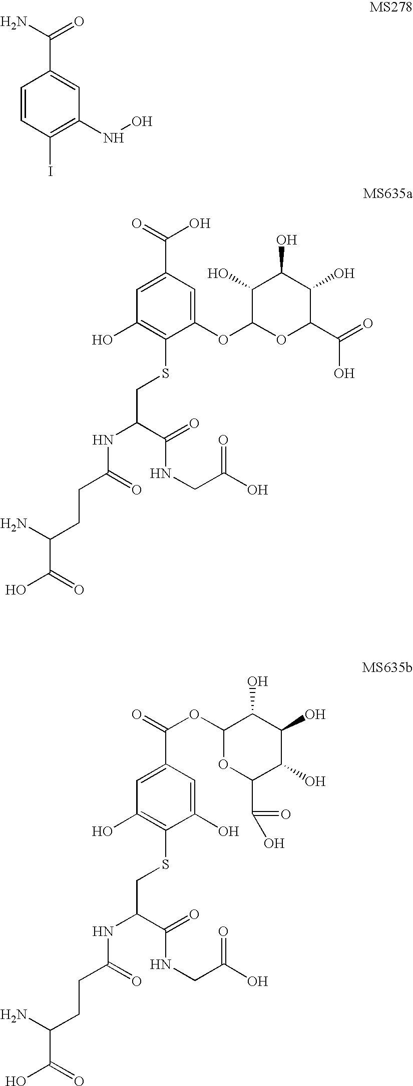

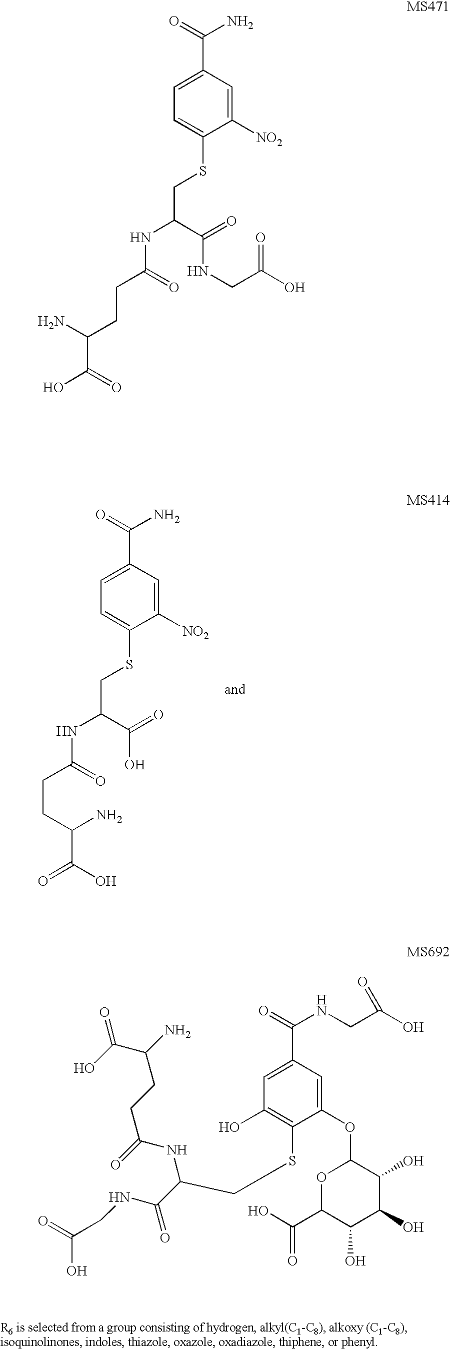

In some embodiments of the kit, the topoisomerase inhibitor is topotecan, irinotecan, lurtotecan, exatecan or a pharmaceutically acceptable salt or metabolite thereof. In some embodiments, the topoisomerase inhibitor is topotecan or a pharmaceutically acceptable salt or metabolite thereof. In some embodiments, the cancer is selected from the group consisting of leukemia, prostate cancer, transitional cell carcinoma of the bladder, pancreatic cancer, colorectal cancer, cervical cancer, and lung cancer. In some embodiments, the kit further comprises an effective amount of a benzopyrone compound of formula (II):

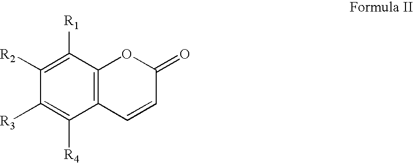

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

In some embodiments, the kit further comprises directions for administering the PARP inhibitor, the topoisomerase inhibitor or both. In some embodiments of the kit, the PARP inhibitor, the topoisomerase inhibitor, or both are in unit dosage form.

### DETAILED DESCRIPTION OF THE INVENTION

### Definitions

“Nitrobenzamide compound(s)” means a compound of the formula (Ia)

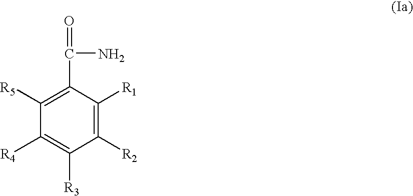

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof. R₁, R₂, R₃, R₄, and R₅ can also be a halide such as chloro, fluoro, or bromo.

“Surgery” means any therapeutic or diagnostic procedure that involves methodical action of the hand or of the hand with an instrument, on the body of a human or other mammal, to produce a curative, remedial, or diagnostic effect.

“Radiation therapy” means exposing a patient to high-energy radiation, including without limitation x-rays, gamma rays, and neutrons. This type of therapy includes without limitation external-beam therapy, internal radiation therapy, implant radiation, brachytherapy, systemic radiation therapy, and radiotherapy.

“Chemotherapy” means the administration of one or more anti-cancer drugs such as, antineoplastic chemotherapeutic agents, chemopreventative agents, and/or other agents to a cancer patient by various methods, including intravenous, oral, intramuscular, intraperitoneal, intravesical, subcutaneous, transdermal, buccal, or inhalation or in the form of a suppository. Chemotherapy may be given prior to surgery to shrink a large tumor prior to a surgical procedure to remove it, after surgery or radiation therapy to prevent the growth of any remaining cancer cells in the body.

The terms “effective amount” or “pharmaceutically effective amount” refer to a sufficient amount of the agent to provide the desired biological, therapeutic, and/or prophylactic result. That result can be reduction and/or alleviation of the signs, symptoms, or causes of a disease, or any other desired alteration of a biological system. For example, an “effective amount” for therapeutic uses is the amount of a nitrobenzamide compound as disclosed herein per se or a composition comprising the nitrobenzamide compound herein required to provide a clinically significant decrease in a disease. An appropriate effective amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

The term “treating” and its grammatical equivalents as used herein include achieving a therapeutic benefit and/or a prophylactic benefit. By therapeutic benefit is meant eradication or amelioration of the underlying disorder being treated. For example, in a cancer patient, therapeutic benefit includes eradication or amelioration of the underlying cancer. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For prophylactic benefit, a method of the invention may be performed on, or a composition of the invention administered to a patient at risk of developing cancer, or to a patient reporting one or more of the physiological symptoms of such conditions, even though a diagnosis of the condition may not have been made.

### Nitrobenzamide Compounds

Compounds useful in the present invention are of Formula (Ia)

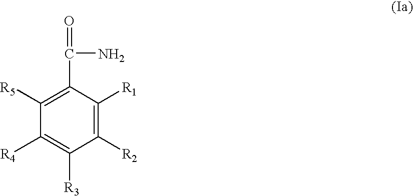

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof. R₁, R₂, R₃, R₄, and R₅ can also be a halide such as chloro, fluoro, or bromo.

A preferred compound of formula Ia is

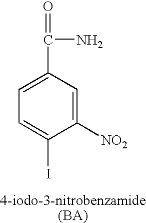

The present invention provides for the use of the aforesaid nitrobenzamide compounds for the treatment of leukemia including acute promyleocytic leukemia in peripheral blood, lung cancer, bladder cancer, colon cancer, rectal cancer, prostate cancer, pancreatic cancer, and cervical cancer, as well as other cancer types described herein (U.S. Pat. No. 5,464,871, U.S. Pat. No. 5,670,518, and U.S. Pat. No. 6,004,978 are incorporated herein by reference in their entirety). The present invention also provides the use of the aforesaid nitrobenzamide compounds for the treatment of Gleevac (Imanitib Mesylate) resistant patient population. Gleevec is a tyrosine kinase inhibitor.

In some preferred embodiments, the nitrobenzamide compounds of the present invention are used for the treatment of cervical cancer. In other embodiments, the nitrobenzamide compounds of the present invention are used for the treatment of lung cancer including small cell lung cancer. In other embodiments, the nitrobenzamide compounds of the present invention are used for the treatment of colon and rectal cancers. In some preferred embodiments, the nitrobenzamide compounds of the present invention are used for the treatment of bladder and prostate cancer. In some preferred embodiments, the nitrobenzamide compounds of the present invention are used for the treatment of liver and pancreatic cancer. In some preferred embodiments, the nitrobenzamide compounds of the present invention are used for the treatment of leukemia, cervical, glioma, and melanoma.

In still further preferred embodiments, the nitrobenzamide compounds of the present invention are used for the treatment of cancers derived from stem cells. In malignancies described herein, a proportion of tumor cells—‘cancer stem cells’—have the capacity for extensive proliferation and transferal of the tumor. An alteration in stem cell fate and growth may play a role in tumorigenesis. Epithelial stem cells have a life-span at least as long as that of the organism, and thus they are thought to be susceptible to multiple genetic hits which cumulatively may result in tumor formation. Many cancers, such as those of the skin and colon, arise in tissues that are constantly replenished with cells throughout life. But the crucial mutations that lead to the disease are likely to have occurred during the tissues' formative period, when cells are dividing exponentially.

The stem cell compartment, now identified virtually in every tissue, can be defined as a subset of rare cells, endowed with the exclusive prerogative of self-renewal and persistence throughout the organism's life, in contrast with differentiated cells, which form the tissue bulk, but usually feature a postmitotic behavior and a short lifespan. The fact that several mutations are necessary for a cell to become cancerous may suggest that in many tissues the mutations may accumulate in stem cells. As cancer stem cells self-renew, it follows that they may be derived either from self-renewing normal stem cells, or from more differentiated cells that acquire peculiar properties of stem cells. Consistently, a tumor can be conceived as a tissue, including both “differentiated” cells, and a subset of “cancer stem cells”, which maintain the tumor mass, and are likely responsible for formation of secondary tumors (metastasis). Hence, nitrobenzamides of the present invention can be used to target cancers derived from stem cells.

In some embodiments, the present invention provides for the use of the aforesaid nitrobenzamide compounds in combination with topoisomerase inhibitors for the treatment of cancer including but not limited to leukemia, lung cancer, bladder cancer, colon cancer, rectal cancer, prostate cancer, pancreatic cancer, and cervical cancer, as well as other cancer types described herein (U.S. Pat. No. 5,464,871, U.S. Pat. No. 5,670,518, and U.S. Pat. No. 6,004,978 are incorporated herein by reference in their entirety). In some embodiments, in order to carry out the current invention, the compositions and methods disclosed in U.S. Pat. No. 7,405,227 can be used. All patents and patent applications are herein incorporated by reference in their entirety.

In some preferred embodiments, the nitrobenzamide compounds in combination with topoisomerase inhibitors are used for the treatment of cervical cancer. In other embodiments, the nitrobenzamide compounds in combination with topoisomerase inhibitors are used for the treatment of lung cancer including small cell lung cancer. In other embodiments, the nitrobenzamide compounds in combination with topoisomerase inhibitors are used for the treatment of colon and rectal cancers. In some preferred embodiments, the nitrobenzamide compounds in combination with topoisomerase inhibitors are used for the treatment of bladder and prostate cancer. In some preferred embodiments, the nitrobenzamide compounds in combination with topoisomerase inhibitors are used for the treatment of liver and pancreatic cancer. In some preferred embodiments, the nitrobenzamide compounds in combination with topoisomerase inhibitors are used for the treatment of leukemia, cervical, glioma, and melanoma. In still further preferred embodiments, the nitrobenzamide compounds in combination with topoisomerase inhibitors are used for the treatment of cancers derived from stem cells. In some embodiments, the nitrobenzamide compound of the invention is 4-iodo-3-nitrobenzamide (BA).

The present invention discloses a non-clinical pharmacology of 4-iodo-3-nitrobenzamide (BA) in human tumor and normal primary cells and also in mice. In vitro BA inhibits the proliferation of a variety of human tumor cells including colon, prostate, cervix, lung, melanoma, lymphoma, and leukemia. In vivo BA in combination with topoisomerase inhibitors, such as topotecan and irinotecan, is evaluated in animal models of carcinogenesis. Once-daily or twice-weekly administration of BA inhibits tumor growth in the human colon, lung, and cervical carcinoma xenograft model in both nude and SCID mice, and positively affects the survival rate of animals exposed to the drug given daily or twice weekly.

It has been reported that nitrobenzamide compounds have selective cytotoxicity upon malignant cancer cells but not upon nonmalignant cancer cells. See Rice et al., Proc. Natl. Acad. Sci. USA 89:7703-7707 (1992). In one embodiment, the nitrobenzamide compounds utilized in the methods of the present invention may exhibit more selective toxicity towards tumor cells than non-tumor cells.

It has been reported that the tumorgenicity of nitrobenzamide and nitrososbenzamide compounds is enhanced when BSO is co-administered to cancer cells. See Mendeleyev et al., Biochemical Pharmacol 50(5):705-714 (1995). Buthionine sulfoximine (BSO) inhibits gamma-glutamylcysteine synthetase, a key enzyme in the biosynthesis of glutathione, which is responsible in part for cellular resistance to chemotherapy. See Chen et al., Chem Biol Interact. April 24; 111-112:263-75 (1998). The invention also provides a method for treating cancer comprising the administration of a nitrobenzamide and/or benzopyrone compound in combination with BSO.

In addition to BSO, other inhibitors of gamma-glutamylcysteine synthetase can be used in combination with nitrobenzamide and/or benzopyrone compounds. Other suitable analogs of BSO include, but are not limited to, proprothionine sulfoximine, methionine sulfoximine, ethionine sulfoximine, methyl buthionine sulfoximine, γ-glutamyl-α-aminobutyrate and γ-glutamylcysteine.

### Benzopyrone Compounds

In some embodiments, the benzamide compounds are administered in combination with benzopyrone compounds of formula II. The benzopyrone compounds of formula II are,

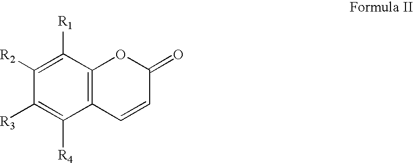

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof (U.S. Pat. No. 5,484,951 is incorporated herein by reference in its entirety).

In a preferred embodiment, the invention relates to the following benzopyrone compound of formula II

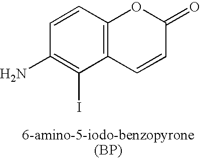

### Mechanism of Nitrobenzamide Compounds

Not intending to be limited by one mechanism of action, the compounds described herein are believed to have anti-cancer properties via the modulation of a poly (ADP-ribose) polymerase enzyme. The drugs' mechanism of action is related to their ability to act as a ligand for the nuclear enzyme poly (ADP-ribose) polymerase (PARP-1). See Mendeleyev et al., supra, (1995). PARP-1 is expressed in the nucleus and catalyzes the conversion of β-nicotinamide adenine dinucleotide (NAD⁺) into nicotinamide and poly-ADP-ribose (PAR). PARP-1's role in homeostatic conditions seems to be limited to DNA transcription and repair. However, when cellular stress causes DNA damage, PARP-1 activity increases dramatically, which appears to be necessary for genomic integrity. Shall et al., Mutat Res. June 30; 460(1):1-15 (2000).

One of PARP-1's functions is to synthesize the biopolymer, poly (ADP-ribose). Both poly (ADP-ribose) and PARP-1 have been linked to the repair of DNA repair, apoptosis, the maintenance of genomic stability, and carcinogenesis. See Masutani et al., Genes, Chromosomes, and Cancer 38:339-348 (2003). PARP-1 plays a role in DNA repair, specifically base excision repair (BER). BER is a protection mechanism in mammalian cells for single-base DNA breakage. PARP-1 binds to the ends of DNA fragments through its zinc finger domains with great affinity and thereby acts as a DNA damage sensor. Gradwohl et al., Proc. Natl. Acad. Sci. USA 87:2990-2994 (1990); Murcia et al., Trends Biochem Sci 19: 172-176 (1994). A breakage in the DNA triggers a binding response by PARP-1 to the site of the break. PARP-1 then increases its catalytic activity several hundred fold (See Simonin et al., J Biol Chem 278: 13454-13461 (1993)) and begins to convert poly ADP-ribosylation of itself (Desmarais et al., Biochim Biophys Acta 1078: 179-186 (1991)) and BER proteins, such as DNA-PKcs and the molecular scaffold protein XRCC-1. See Ruscetti et al., J. Biol. Chem. June 5; 273(23):14461-14467 (1998) and Masson et al., Mol Cell Biol. June; 18(6):3563-71 (1998). BER proteins are rapidly recruited to the site of DNA damage. El-Kaminsy et al., Nucleic Acid Res. 31(19):5526-5533 (2003); Okano et al., Mol Cell Biol. 23(11):3974-3981 (2003). PARP-1's dissociates from the DNA breakage site but it remains in the vicinity of the DNA repair event.

Inhibiting the activity of a PARP molecule includes reducing the activity of these molecules. The term “inhibits” and its grammatical conjugations, such as “inhibitory,” is not intended to require complete reduction in PARP activity. Such reduction is preferably by at least about 50%, at least about 75%, at least about 90%, and more preferably by at least about 95% of the activity of the molecule in the absence of the inhibitory effect, e.g., in the absence of an inhibitor, such as a nitrobenzamide compound of the invention. Most preferably, the term refers to an observable or measurable reduction in activity. In treatment scenarios, preferably the inhibition is sufficient to produce a therapeutic and/or prophylactic benefit in the condition being treated. The phrase “does not inhibit” and its grammatical conjugations does not require a complete lack of effect on the activity. For example, it refers to situations where there is less than about 20%, less than about 10%, and preferably less than about 5% of reduction in PARP activity in the presence of an inhibitor such as a nitrobenzamide compound of the invention.

BA Metabolites:

As used herein “BA” means 4-iodo-3-nitrobenzamide; “BNO” means 4-iodo-3-nitrosobenzamide; “BNHOH” means 4-iodo-3-hydroxyaminobenzamide.

Precursor compounds useful in the present invention are of Formula (Ia)

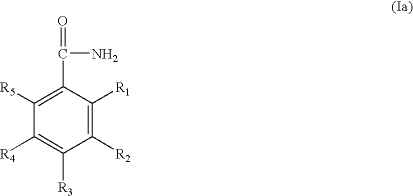

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or pro-drugs thereof. R₁, R₂, R₃, R₄, and R₅ can also be a halide such as chloro, fluoro, or bromo substituents.

A preferred precursor compound of formula Ia is:

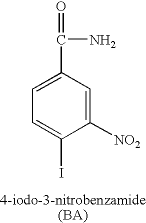

Some metabolites useful in the present invention are of the Formula (IIa):

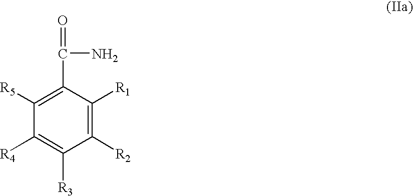

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

The following compositions are preferred metabolite compounds, each represented by a chemical formula:

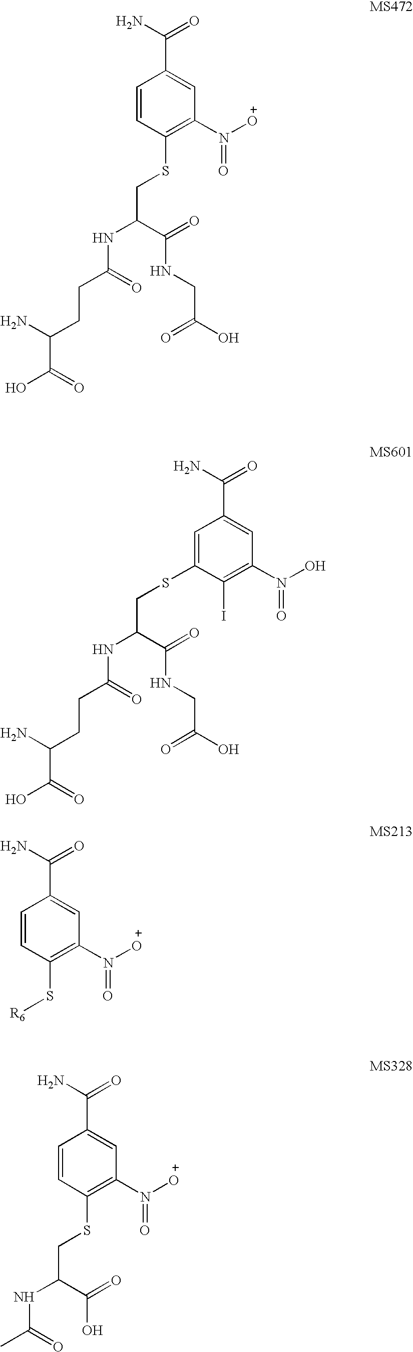

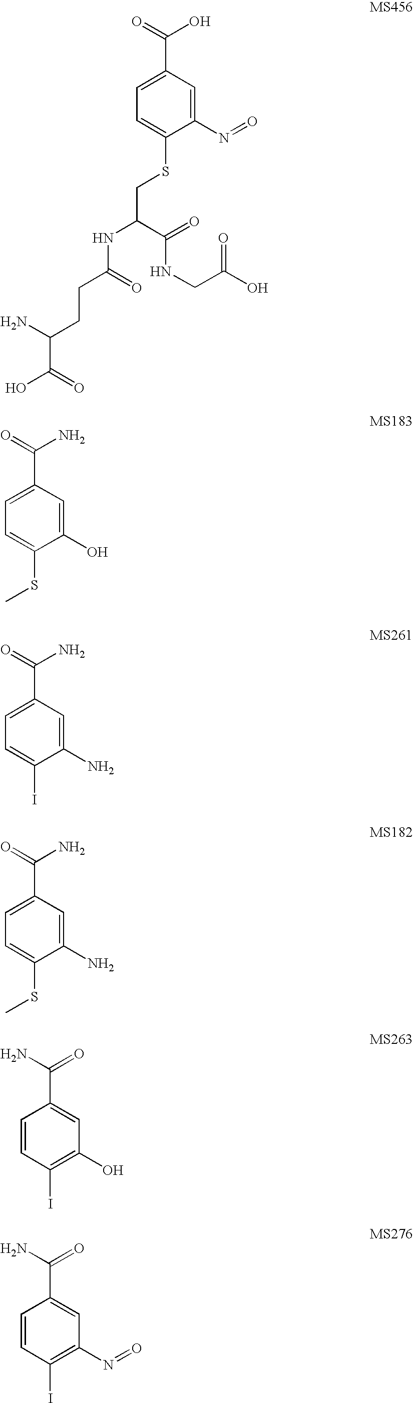

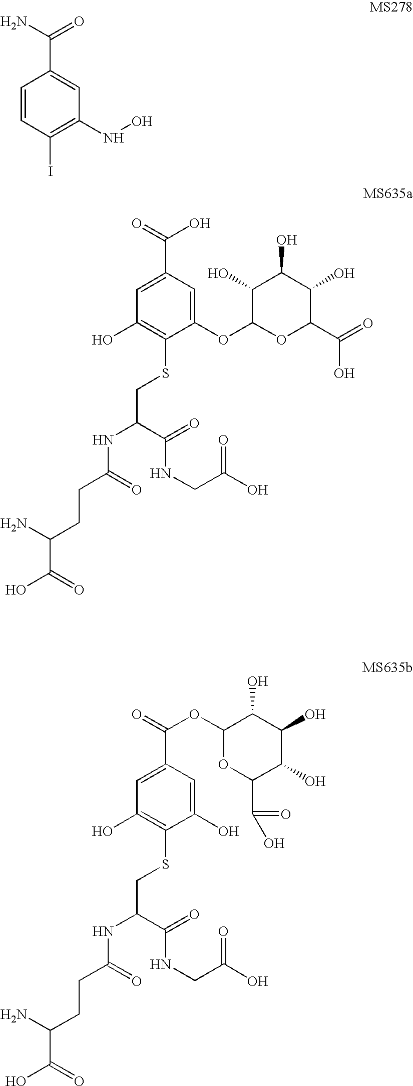

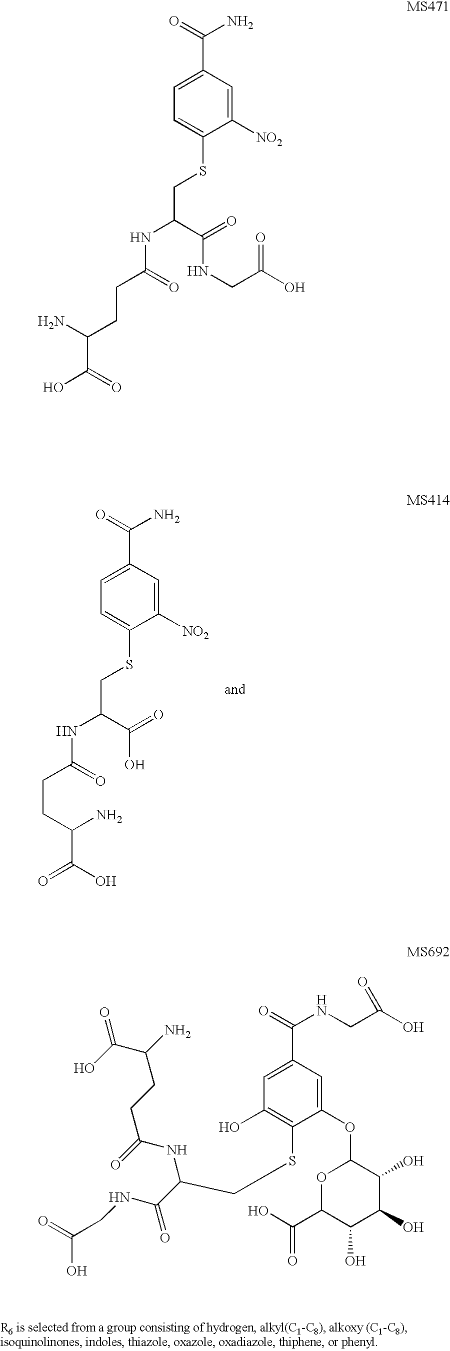

While not being limited to any one particular mechanism, the following provides an example for MS292 metabolism via a nitroreductase or glutathione conjugation mechanism:

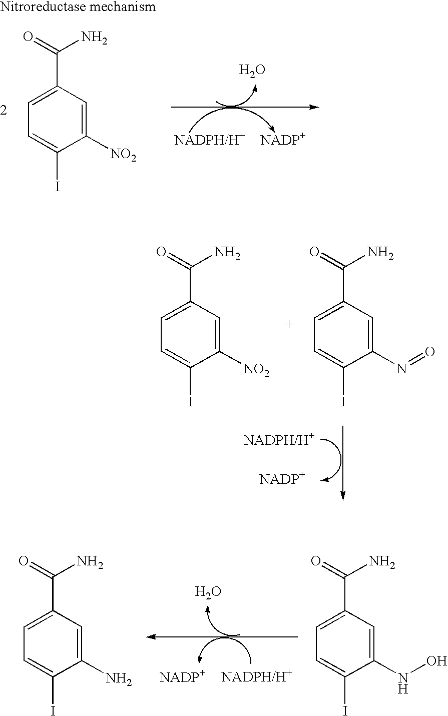

BA glutathione conjugation and metabolism:

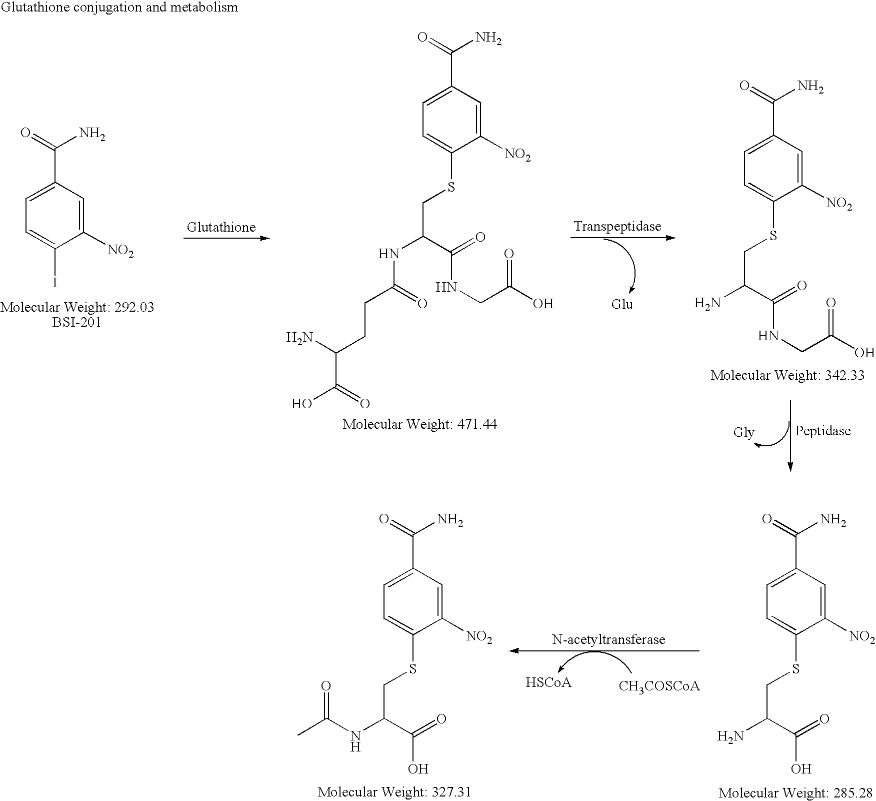

It has been reported that nitrobenzamide metabolite compounds have selective cytotoxicity upon malignant cancer cells but not upon non-malignant cancer cells. See Rice et al., Proc. Natl. Acad. Sci. USA 89:7703-7707 (1992), incorporated herein in it entirety. In one embodiment, the nitrobenzamide metabolite compounds utilized in the methods of the present invention may exhibit more selective toxicity towards tumor cells than non-tumor cells. The metabolites according to the invention may thus be administered to a patient in need of such treatment in conjunction with chemotherapy with at least one topoisomerase inhibitor. The dosage range for such metabolites may be in the range of about 0.0004 to about 0.5 mmol/kg (millimoles of metabolite per kilogram of patient body weight), which dosage corresponds, on a molar basis, to a range of about 0.1 to about 100 mg/kg of BA. Other effective ranges of dosages for metabolites are 0.0024-0.5 mmol/kg and 0.0048-0.25 mmol/kg. Such doses may be administered on a daily, every-other-daily, twice-weekly, weekly, bi-weekly, monthly or other suitable schedule. Essentially the same modes of administration may be employed for the metabolites as for BA—e.g. oral, i.v., i.p., etc.

Topoisomerase Inhibitors

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. Topoisomerases have become popular targets for cancer chemotherapy treatments. It is thought that topoisomerase inhibitors block the ligation step of the cell cycle, generating single and double stranded breaks that harm the integrity of the genome. Introduction of these breaks subsequently lead to apoptosis and cell death. Topoisomerase inhibitors are often divided according to which type of enzyme it inhibits. Topoisomerase I, the type of topoisomerase most often found in eukaryotes, is targeted by topotecan, irinotecan, lurtotecan and exatecan, each of which is commercially available from. Topotecan is available from GlaxoSmithKline under the trade name Hycamtim®. Irinotecan is available from Pfizer under the trade name Camptosar®. Lurtotecan may be obtained as a liposomal formulation from Gilead Sciences Inc. Topoisomerase inhibitors may be administered at an effective dose. In some embodiments an effective dose for treatment of a human will be in the range of about 0.01 to about 10 mg/m²/day. The treatment may be repeated on a daily, bi-weekly, semi-weekly, weekly, or monthly basis. In some embodiments, a treatment period may be followed by a rest period of from one day to several days, or from one to several weeks. In combination with a PARP-1 inhibitor, the PARP-1 inhibitor and the topoisomerase inhibitor may be dosed on the same day or may be dosed on separate days.

Compounds that target type II topoisomerase are split into two main classes: topoisomerase poisons, which target the topoisomerase-DNA complex, and topoisomerase inhibitors, which disrupt catalytic turnover. Topo II poisons include but are not limited to eukaryotic type II topoisomerase inhibitors (topo II): amsacrine, etoposide, etoposide phosphate, teniposide and doxorubicin. These drugs are anti-cancer therapies. Examples of topoisomerase inhibitors include ICRF-193. These inhibitors target the N-terminal ATPase domain of topo II and prevent topo II from turning over. The structure of this compound bound to the ATPase domain has been solved by Classen (Proceedings of the National Academy of Science, 2004) showing that the drug binds in a non-competitive manner and locks down the dimerization of the ATPase domain.

Irinotecan

Irinotecan is a topoisomerase 1 inhibitor. Chemically, it is a semisynthetic analogue of the natural alkaloid camptothecin. Its main use is in colon cancer, particularly in combination with other chemotherapy agents. This includes the regimen FOLFIRI which consists of infusional 5-fluorouracil, leucovorin, and irinotecan.

Irinotecan is activated by hydrolysis to SN-38, an inhibitor of topoisomerase I. This is then inactivated by glucuronidation by uridine diphosphate glucoronosyltransferase 1A1 (UGT1A1). The inhibition of topoisomerase I by the active metabolite SN-38 eventually leads to inhibition of both DNA replication and transcription.

Topotecan:

Topotecan hydrochloride (trade name Hycamtin) is a topoisomerase 1 inhibitor. Topotecan hydrochloride is approved by the Food and Drug Administration (FDA) to treat ovarian cancer and small cell lung cancer in patients whose cancer has not gotten better with earlier chemotherapy. It is also approved to be used together with cisplatin, a platinum compound, to treat cervical cancer in some women whose cancer has not gotten better or has recurred. Topotecan hydrochloride is also being studied in the treatment of other types of cancer. Topotecan can be administered via intravenous injection or orally.

Clinical Efficacy:

Clinical efficacy may be measured by any method known in the art. In some embodiments, clinical efficacy of the combination of topoisomerase inhibitor and PARP-1 inhibitor (e.g. topotecan and BA) may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD≧6 months. The CBR for combination therapy with a topoisomerase inhibitor and a PARP-1 inhibitor (e.g. topotecan and BA; CBR_{T-B}) may be compared to that of therapy with topotecan alone (CBR_{T}). In some embodiments, CBR_{T-B} is at least about 40%, at least about 50% or at least about 60%.

In some embodiments disclosed herein, the methods include predetermining that a cancer is treatable by PARP modulators. Some such methods comprise identifying a level of PARP in a tumor sample of a patient, determining whether the level of PARP expression in the sample is greater than a predetermined value, and, if the PARP expression is greater than said predetermined value, treating the patient with a combination of a topoisomerase inhibitor (such as topotecan or irinotecan) and a PARP-1 inhibitor such as BA.

PARP inhibitors kill cells where this form of DNA repair is absent; and thus are effective in killing BRCA deficient tumor cells and other similar tumor cells. Normal cells may be unaffected by the drug as they may still possess this DNA repair mechanism. This treatment might also be applicable to other forms of cancer that behave like BRCA deficient cancer. In some embodiments, an advantage of treating with PARP inhibitors is that it is targeted therapy: tumor cells are killed while normal cells appear unaffected. This is because PARP inhibitors exploit the specific genetic make-up of some tumor cells.

Patients deficient in BRCA genes have up-regulated levels of PARP. PARP up-regulation may be an indicator of other defective DNA-repair pathways and unrecognized BRCA-like genetic defects. Assessment of PARP-1 gene expression is an indicator of tumor sensitivity to PARP inhibitor. The BRCA deficient patients treatable by PARP inhibitors can be identified if PARP is up-regulated. Further, such BRCA deficient patients can be treated with PARP inhibitors.

In some embodiments, a sample is collected from a patient having a lesion suspected of being cancerous. While such sample may be any available biological tissue, in most cases the sample will be a portion of the suspected lesion, whether obtained by laparoscopy or open surgery. PARP expression may then be analyzed and, if the PARP expression is above a predetermined level (e.g. is up-regulated vis-á-vis normal tissue) the patient may be treated with a PARP-1 inhibitor in combination with a topoisomerase inhibitor.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer may be treated with a PARP inhibitor in combination with a topoisomerase inhibitor.

Tumors that have deficiency in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to many cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors kill cells where this form of DNA repair is absent; and thus are effective in killing BRCA deficient tumor cells and other similar tumor cells. Normal cells may be unaffected by the drug as they may still possess this DNA repair mechanism. In some embodiments, an advantage of treating with PARP inhibitors is that it is targeted therapy: tumor cells are killed while normal cells appear unaffected. This is because PARP inhibitors exploit the specific genetic make-up of some tumor cells. While not wishing to be bound by theory, it is thought that combined treatment with PARP inhibitor and a topoisomerase inhibitor may permit efficacious dosing of the topoisomerase inhibitor at a lower, and hence less toxic, dose. In some embodiments, the effective dose of topoisomerase inhibitor used with a PARP inhibitor may be about 10 to about 90%, about 10 to about 80%, about 10 to about 60%, about 10 to about 50%, less than about 90%, less than about 80%, less than about 60%, less than about 50% or less than about 40% of an effective dose of the topoisomerase inhibitor used alone.

### Sample Collection, Preparation and Separation

Biological samples may be collected from a variety of sources from a patient including a body fluid sample, or a tissue sample. Samples collected can be human normal and tumor samples, nipple aspirants. The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of PARP. Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermeable membrane in a process in which ions are transported through semi-permeable membranes from one solution to another under the influence of a potential gradient. Since the membranes used in electrodialysis may have the ability to selectively transportions having positive or negative charge, reject ions of the opposite charge, or to allow species to migrate through a semipermeable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC) etc.

Identifying Level of PARP

The poly (ADP-ribose) polymerase (PARP) is also known as poly (ADP-ribose) synthase and poly ADP-ribosyltransferase. PARP catalyzes the formation of poly (ADP-ribose) polymers which can attach to nuclear proteins (as well as to itself) and thereby modify the activities of those proteins. The enzyme plays a role in DNA repair, but it also plays a role in regulating chromatin in the nuclei (for review see: D. D'amours et al. “Poly (ADP-ribosylation reactions in the regulation of nuclear functions,” Biochem. J. 342: 249-268 (1999)).

PARP-1 comprises an N-terminal DNA binding domain, an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase β, X-ray repair cross-complementing factor-1 (XRCC 1) and PARP-1 itself interact with PARP-1 in this domain. The automodification domain contains a BRCT motif, one of the protein interaction modules. This motif is originally found in the C-terminus of BRCA1 (breast cancer 1, early onset) and is present in various proteins related to DNA repair, recombination and cell-cycle checkpoint control. POU-homeodomain-containing octamer transcription factor-1 (Oct-1), Yin Yang (YY) 1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

In some embodiments, the level of PARP in a sample from a patient is compared to predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a pre-determined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. A pre-determined PARP level may be determined in populations of patients with or without cancer. The predetermined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual. Furthermore, the pre-determined PARP level can be a level determined for each patient individually. The pre-determined PARP level can be any suitable standard. For example, the predetermined PARP level can be obtained from the same or a different human for whom a patient selection is being assessed. In one embodiment, the pre-determined PARP level can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

The analysis of PARP levels in patients is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

In monitoring a patient's PARP levels, over a period of time, which may be days, weeks, months, and in some cases, years, or various intervals thereof, the patient's body fluid sample, e.g., serum or plasma, can be collected at intervals, as determined by the practitioner, such as a physician or clinician, to determine the levels of PARP, and compared to the levels in normal individuals over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring.

### Therapeutic Use of PARP Inhibitors and Topoisomerase Inhibitors

### Cancer Types

The invention provides methods to treat several specific cancers or tumors. For example, cancer types include adrenal cortical cancer, anal cancer, aplastic anemia, bile duct cancer, bladder cancer, bone cancer, bone metastasis, Adult CNS brain tumors, Children CNS brain tumors, Castleman disease, cervical cancer, Childhood Non-Hodgkin's lymphoma, colon and rectum (colorectal) cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, vaginal cancer, vulvar cancer, Waldenstrom's macroglobulinemia, cancers of viral origin and virus-associated cancers.

Carcinoma of the thyroid gland is the most common malignancy of the endocrine system. Carcinoma of the thyroid gland include differentiated tumors (papillary or follicular) and poorly differentiated tumors (medullary or anaplastic). Carcinomas of the vagina include squamous cell carcinoma, adenocarcinoma, melanoma and sarcoma. Testicular cancer is broadly divided into seminoma and nonseminoma types.

Thymomas are epithelial tumors of the thymus, which may or may not be extensively infiltrated by nonneoplastic lymphocytes. The term thymoma is customarily used to describe neoplasms that show no overt atypia of the epithelial component. A thymic epithelial tumor that exhibits clear-cut cytologic atypia and histologic features no longer specific to the thymus is known as a thymic carcinoma (also known as type C thymoma).

The methods provided by the invention may comprise the administration of the benzamide compounds with topoisomerase inhibitors in combination with other therapies. The choice of therapy that can be co-administered with the compositions of the invention will depend, in part, on the condition being treated. For example, for treating acute myleoid leukemia, a benzamide compound of some embodiments of the invention can be used in combination with radiation therapy, monoclonal antibody therapy, chemotherapy, bone marrow transplantation, gene therapy, DNA/RNA therapy, adjuvant therapy, nanotherapy, neoadjuvant therapy, immunotherapy, or a combination thereof.

### Cervical Cancer

In another aspect, the invention provides a method of treating cervical cancer, preferably an adenocarcinoma in the cervix epithelial. Two main types of this cancer exist: squamous cell carcinoma and adenocarcinomas. The former constitutes about 80-90% of all cervical cancers and develops where the ectocervix (portion closest to the vagina) and the endocervix (portion closest to the uterus) join. The latter develop in the mucous-producing gland cells of the endocervix. Some cervical cancers have characteristics of both of these and are called adenosquamous carcinomas or mixed carcinomas.

The chief treatments available for cervical cancer are surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy. Anti-cancer drugs that may be administered as part of chemotherapy to treat cervical cancer include cisplatin, carboplatin, hydroxyurea, irinotecan, bleomycin, vincrinstine, mitomycin, ifosfamide, fluorouracil, etoposide, methotrexate, and combinations thereof.

The methods provided by the invention can provide a beneficial effect for cervical cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Prostate Cancer

In one other aspect, the invention provides methods to treat prostate cancer, preferably a prostate cancer selected from the following: an adenocarcinoma or an adenocarinoma that has migrated to the bone. Prostate cancer develops in the prostate organ in men, which surrounds the first part of the urethra. The prostate has several cell types but 99% of tumors are adenocarcinomas that develop in the glandular cells responsible for generating seminal fluid.

Surgery, immunotherapy, radiation therapy, cryosurgery, hormone therapy, and chemotherapy are some treatments available for prostate cancer patients. Possible surgical procedures to treat prostate cancer include radical retropubic prostatectomy, a radical perineal prostatectomy, and a laparoscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells.

Hormone therapy, also called androgen deprivation therapy or androgen suppression therapy, may be used to treat prostate cancer. Several methods of this therapy are available including an orchiectomy in which the testicles, where 90% of androgens are produced, are removed. Another method is the administration of luteinizing hormone-releasing hormone (LHRH) analogs to lower androgen levels. The LHRH analogs available include leuprolide, goserelin, triptorelin, and histrelin. An LHRH antagonist may also be administered, such as abarelix.

Treatment with an anti-androgen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB).

Chemotherapy may be appropriate where a prostate tumor has spread outside the prostate gland and hormone treatment is not effective. Anti-cancer drugs such as doxorubicin, estramustine, etoposide, mitoxantrone, vinblastine, paclitaxel, docetaxel, carboplatin, and prednisone may be administered to slow the growth of prostate cancer, reduce symptoms and improve the quality of life.

The methods provided by the invention can provide a beneficial effect for prostate cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Pancreatic Cancer

Some embodiments provide methods of treating pancreatic cancer, preferably a pancreatic cancer selected from the following: an epitheliod carcinoma in the pancreatic duct tissue and an adenocarcinoma in a pancreatic duct.

The most common type of pancreatic cancer is an adenocarcinoma, which occurs in the lining of the pancreatic duct. The possible treatments available for pancreatic cancer are surgery, immunotherapy, radiation therapy, and chemotherapy. Possible surgical treatment options include a distal or total pancreatectomy and a pancreaticoduodenectomy (Whipple procedure).

Radiation therapy may be an option for pancreatic cancer patients, specifically external beam radiation where radiation is focused on the tumor by a machine outside the body. Another option is intraoperative electron beam radiation administered during an operation.

Chemotherapy may be used to treat pancreatic cancer patients. Appropriate anti-cancer drugs include 5-fluorouracil (5-FU), mitomycin, ifosfamide, doxorubicin, streptozocin, chlorozotocin, and combinations thereof.

The methods provided by the invention can provide a beneficial effect for pancreatic cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Bladder Cancer

Some embodiments provide methods of treating bladder cancer, preferably a transitional cell carcinoma in urinary bladder. Bladder cancers are urothelial carcinomas (transitional cell carcinomas) or tumors in the urothelial cells that line the bladder. The remaining cases of bladder cancer are squamous cell carcinomas, adenocarcinomas, and small cell cancers. Several subtypes of urothelial carcinomas exist depending on whether they are noninvasive or invasive and whether they are papillary, or flat. Noninvasive tumors are in the urothelium, the innermost layer of the bladder, while invasive tumors have spread from the urothelium to deeper layers of the bladder's main muscle wall. Invasive papillary urothelial carcinomas are slender finger-like projections that branch into the hollow center of the bladder and also grow outward into the bladder wall. Non-invasive papillary urothelial tumors grow towards the center of the bladder. While a non-invasive, flat urothelial tumor (also called a flat carcinoma in situ) is confined to the layer of cells closest to the inside hollow part of the bladder, an invasive flat urothelial carcinoma invades the deeper layer of the bladder, particularly the muscle layer.

To treat bladder cancer, surgery, radiation therapy, immunotherapy, chemotherapy, or a combination thereof may be applied. Some possible surgical options are a transurethral resection, a cystectomy, or a radical cystectomy. Radiation therapy for bladder cancer may include external beam radiation and brachytherapy.

Immunotherapy is another method that may be used to treat a bladder cancer patient. Typically this is accomplished intravesically, which is the administration of a treatment agent directly into the bladder by way of a catheter. One method is Bacillus Calmete-Guerin (BCG) where a bacterium sometimes used in tuberculosis vaccination is given directly to the bladder through a catheter. The body mounts an immune response to the bacterium, thereby attacking and killing the cancer cells.

Another method of immunotherapy is the administration of interferons, glycoproteins that modulate the immune response. Interferon alpha is often used to treat bladder cancer.

Anti-cancer drugs that may be used in chemotherapy to treat bladder cancer include thitepa, methotrexate, vinblastine, doxorubicin, cyclophosphamide, paclitaxel, carboplatin, cisplatin, ifosfamide, gemcitabine, or combinations thereof.

The methods provided by the invention can provide a beneficial effect for bladder cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Colon Cancer and Rectal Cancer

In another aspect, the invention provides methods to treat colorectal cancers. In some embodiments, the method comprises administering benzopyrone compounds alone into a subject. In other embodiments, the method comprises administering benzopyrone compounds in combination with one or more anti-tumor agents as listed herein into a subject.

Colorectal cancer includes cancerous growths in the colon, rectum and appendix. Many clorectal cancers are thought to arise from adenomatous polyps in the colon. Colorectal cancer originates from the epithelial cells lining the gastrointestinal tract. Hereditary or somatic mutations in specific DNA sequences, among which are included DNA replication or DNA repair genes, and also the APC, K-Ras, NOD2 and p53 genes, lead to unrestricted cell division. Therapy is usually through surgery, which in many cases is followed by chemotherapy. Bacillus Calmette-Guérin (BCG) is being investigated as an adjuvant mixed with autologous tumor cells in immunotherapy for colorectal cancer.

Over 20% of patients present with metastatic (stage 1V) colorectal cancer at the time of diagnosis, and up to 25% of this group have isolated liver metastasis that is potentially resectable. Patients with colon cancer and metastatic disease to the liver may be treated in either a single surgery or in staged surgeries depending upon the fitness of the patient for prolonged surgery, the difficulty expected with the procedure with either the colon or liver resection, and the comfort of the surgery performing potentially complex hepatic surgery.

The methods provided by the invention can provide a beneficial effect for colorectal cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Acute Myeloid Leukemia

Some embodiments provide methods of treating acute myeloid leukemia (AML), preferably acute promyleocytic leukemia in peripheral blood. AML begins in the bone marrow but can spread to other parts of the body including the lymph nodes, liver, spleen, central nervous system, and testes. It is acute meaning it develops quickly and may be fatal if not treated within a few months. AML is characterized by immature bone marrow cells usually granulocytes or monocytes, which continue to reproduce and accumulate.

AML may be treated by immunotherapy, radiation therapy, chemotherapy, bone marrow or peripheral blood stem cell transplantation, or a combination thereof. Radiation therapy includes external beam radiation and may have side effects. Anti-cancer drugs that may be used in chemotherapy to treat AML include cytarabine, anthracycline, anthracenedione, idarubicin, daunorubicin, idarubicin, mitoxantrone, thioguanine, vincristine, prednisone, etoposide, or a combination thereof.

Monoclonal antibody therapy may be used to treat AML patients. Small molecules or radioactive chemicals may be attached to these antibodies before administration to a patient in order to provide a means of killing leukemia cells in the body. The monoclonal antibody, gemtuzumab ozogamicin, which binds CD33 on AML cells, may be used to treat AML patients unable to tolerate prior chemotherapy regimens.

Bone marrow or peripheral blood stem cell transplantation may be used to treat AML patients. Some possible transplantation procedures are an allogenic or an autologous transplant.

The methods provided by the invention can provide a beneficial effect for leukemia patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

There are other types of leukemia's that can also be treated by the methods provided by the invention including but not limited to, Acute Lymphocytic Leukemia, Acute Myeloid Leukemia, Chronic Lymphocytic Leukemia, Chronic Myeloid Leukemia, Hairy Cell Leukemia, Myelodysplasia, and Myeloproliferative Disorders.

### Lung Cancer

Some embodiments provide methods to treat lung cancer. The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer accounts for 15-20% of lung cancers.

Small cell lung cancer is a disease in which malignant (cancer) cells form in the tissues of the lung. There are three types of small cell lung cancer. These three types include many different types of cells. The cancer cells of each type grow and spread in different ways. The types of small cell lung cancer are named for the kinds of cells found in the cancer and how the cells look when viewed under a microscope: small cell carcinoma (oat cell cancer); mixed small cell/large cell carcinoma; and combined small cell carcinoma. For most patients with small cell lung cancer, current treatments do not cure the cancer.

Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy.

Some anti-cancer drugs that may be used in chemotherapy to treat lung cancer include cisplatin, carboplatin, paclitaxel, docetaxel, gemcitabine, vinorelbine, irinotecan, etoposide, vinblastine, gefitinib, ifosfamide, methotrexate, or a combination thereof. Photodynamic therapy (PDT) may be used to treat lung cancer patients.

The methods described herein can provide a beneficial effect for lung cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Skin Cancer

Some embodiments provide methods of treating skin cancer. There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body. Different types of treatment are available for patients with non-melanoma and melanoma skin cancer and actinic keratosis including surgery, radiation therapy, chemotherapy and photodynamic therapy. Some possible surgical options for treatment of skin cancer are mohs micrographic surgery, simple excision, electrodesiccation and curettage, cryosurgery, laser surgery. Radiation therapy may be external beam radiation therapy or brachytherapy. Other types of treatments that are being tested in clinical trials are biologic therapy or immunotherapy, chemoimmunotherapy, topical chemotherapy with fluorouracil and photodynamic therapy.

The methods provided by the invention can provide a beneficial effect for skin cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Eye Cancer, Retinoblastoma

Some embodiments provide methods to treat eye retinoblastoma. Retinoblastoma is a malignant tumor of the retina. Although retinoblastoma may occur at any age, it most often occurs in younger children, usually before the age of 5 years. The tumor may be in one eye only or in both eyes. Retinoblastoma is usually confined to the eye and does not spread to nearby tissue or other parts of the body. Treatment options that attempt to cure the patient and preserve vision include enucleation (surgery to remove the eye), radiation therapy, cryotherapy, photocoagulation, immunotherapy, thermotherapy and chemotherapy. Radiation therapy may be external beam radiation therapy or brachytherapy.

The methods provided by the invention can provide a beneficial effect for eye retinoblastoma patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Eye Cancer, Intraocular Melanoma

Some embodiments provide methods to treat intraocular (eye) melanoma. Intraocular melanoma, a rare cancer, is a disease in which cancer cells are found in the part of the eye called the uvea. The uvea includes the iris, the ciliary body, and the choroid. Intraocular melanoma occurs most often in people who are middle aged. Treatments for intraocular melanoma include surgery, immunotherapy, radiation therapy and laser therapy. Surgery is the most common treatment of intraocular melanoma. Some possible surgical options are iridectomy, iridotrabeculectomy, iridocyclectomy, choroidectomy, enucleation and orbital exenteration. Radiation therapy may be external beam radiation therapy or brachytherapy. Laser therapy may be an intensely powerful beam of light to destroy the tumor, thermotherapy or photocoagulation.

The methods provided by the invention can provide a beneficial effect for intraocular melanoma patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Liver Cancer

Some embodiments provide methods to treat primary liver cancer (cancer that begins in the liver). Primary liver cancer can occur in both adults and children. Different types of treatments are available for patients with primary liver cancer. These include surgery, immunotherapy, radiation therapy, chemotherapy and percutaneous ethanol injection. The types of surgery that may be used are cryosurgery, partial hepatectomy, total hepatectomy and radiofrequency ablation. Radiation therapy may be external beam radiation therapy, brachytherapy, radiosensitizers or radiolabel antibodies. Other types of treatment include hyperthermia therapy and immunotherapy.

The methods provided by the invention can provide a beneficial effect for liver cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Kidney Cancer

Some embodiments provide methods to treat kidney cancer. Kidney cancer (also called renal cell cancer or renal adenocarcinoma) is a disease in which malignant cells are found in the lining of tubules in the kidney. Kidney cancer may be treated by surgery, radiation therapy, chemotherapy and immunotherapy. Some possible surgical options to treat kidney cancer are partial nephrectomy, simple nephrectomy and radical nephrectomy. Radiation therapy may be external beam radiation therapy or brachytherapy. Stem cell transplant may be used to treat kidney cancer.

The methods provided by the invention can provide a beneficial effect for kidney cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Thyroid Cancer

Some embodiments provide methods of treating thyroid cancer. Thyroid cancer is a disease in which cancer (malignant) cells are found in the tissues of the thyroid gland. The four main types of thyroid cancer are papillary, follicular, medullary and anaplastic. Thyroid cancer may be treated by surgery, immunotherapy, radiation therapy, hormone therapy and chemotherapy. Surgery is the most common treatment of thyroid cancer. Some possible surgical options for treatment of thyroid cancer are lobectomy, near-total thyroidectomy, total thyroidectomy and lymph node dissection. Radiation therapy may be external radiation therapy or may required intake of a liquid that contains radioactive iodine. Hormone therapy uses hormones to stop cancer cells from growing. In treating thyroid cancer, hormones can be used to stop the body from making other hormones that might make cancer cells grow.

The methods provided by the invention can provide a beneficial effect for thyroid cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### AIDS Related Cancers

AIDS-Related Lymphoma

Some embodiments provide methods of treating AIDS-related lymphoma. AIDS-related lymphoma is a disease in which malignant cells form in the lymph system of patients who have acquired immunodeficiency syndrome (AIDS). AIDS is caused by the human immunodeficiency virus (HIV), which attacks and weakens the body's immune system. The immune system is then unable to fight infection and diseases that invade the body. People with HIV disease have an increased risk of developing infections, lymphoma, and other types of cancer. Lymphomas are cancers that affect the white blood cells of the lymph system. Lymphomas are divided into two general types: Hodgkin's lymphoma and non-Hodgkin's lymphoma. Both Hodgkin's lymphoma and non-Hodgkin's lymphoma may occur in AIDS patients, but non-Hodgkin's lymphoma is more common. When a person with AIDS has non-Hodgkin's lymphoma, it is called an AIDS-related lymphoma. Non-Hodgkin's lymphomas may be indolent (slow-growing) or aggressive (fast-growing). AIDS-related lymphoma is usually aggressive. The three main types of AIDS-related lymphoma are diffuse large B-cell lymphoma, B-cell immunoblastic lymphoma and small non-cleaved cell lymphoma.

Treatment of AIDS-related lymphoma combines treatment of the lymphoma with treatment for AIDS. Patients with AIDS have weakened immune systems and treatment can cause further damage. For this reason, patients who have AIDS-related lymphoma are usually treated with lower doses of drugs than lymphoma patients who do not have AIDS. Highly-active antiretroviral therapy (HAART) is used to slow progression of HIV. Medicine to prevent and treat infections, which can be serious, is also used. AIDS-related lymphomas may be treated by chemotherapy, immunotherapy, radiation therapy and high-dose chemotherapy with stem cell transplant. Radiation therapy may be external beam radiation therapy or brachytherapy. AIDS-related lymphomas can be treated by monoclonal antibody therapy.

The methods provided by the invention can provide a beneficial effect for AIDS-related lymphoma patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

Kaposi's Sarcoma

Some embodiments provide methods of treating Kaposi's sarcoma. Kaposi's sarcoma is a disease in which cancer cells are found in the tissues under the skin or mucous membranes that line the mouth, nose, and anus. Classic Kaposi's sarcoma usually occurs in older men of Jewish, Italian, or Mediterranean heritage. This type of Kaposi's sarcoma progresses slowly, sometimes over 10 to 15 years. Kaposi's sarcoma may occur in people who are taking immunosuppressants. Kaposi's sarcoma in patients who have Acquired Immunodeficiency Syndrome (AIDS) is called epidemic Kaposi's sarcoma. Kaposi's sarcoma in people with AIDS usually spreads more quickly than other kinds of Kaposi's sarcoma and often is found in many parts of the body. Kaposi's sarcoma may be treated with surgery, chemotherapy, radiation therapy and immunotherapy. External radiation therapy is a common treatment of Kaposi's sarcoma. Some possible surgical options to treat Kaposi's Sarcoma are local excision, electrodessication and curettage, and cryotherapy.

The methods provided by the invention can provide a beneficial effect for Kaposi's sarcoma, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Viral-Induced Cancers

Some embodiments provide methods of treating viral-induced cancers. Several common viruses are clearly or probable causal factors in the etiology of specific malignancies. These viruses either normally establish latency or few can become persistent infections. Oncogenesis is probably linked to an enhanced level of viral activation in the infected host, reflecting heavy viral dose or compromised immune control. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In general, these malignancies occur relatively early in life, typically peaking in middle-age or earlier.

Virus-Induced Hepatocellular Carcinoma

The causal relationship between both HBV and HCV and hepatocellular carcinoma or liver cancer is established through substantial epidemiologic evidence. Both appear to act via chronic replication in the liver by causing cell death and subsequent regeneration. Different types of treatments are available for patients with liver cancer. These include surgery, immunotherapy, radiation therapy, chemotherapy and percutaneous ethanol injection. The types of surgery that may be used are cryosurgery, partial hepatectomy, total hepatectomy and radiofrequency ablation. Radiation therapy may be external beam radiation therapy, brachytherapy, radiosensitizers or radiolabel antibodies. Other types of treatment include hyperthermia therapy and immunotherapy.

The methods provided by the invention can provide a beneficial effect for virus induce hepatocellular carcinoma patients, by administration of a nitrobenzamide compound with a topoisomerase inhibitor or a combination of administration of a nitrobenzamide compound with a topoisomerase inhibitor and surgery, radiation therapy, chemotherapy, percutaneous ethanol injection, hyperthermia therapy and immunotherapy, or a combination thereof.

Viral-Induced Adult T Cell Leukemia/Lymphoma

The association between HTLV-1 and Adult T cell leukemia (ATL) is firmly established. Unlike the other oncogenic viruses found throughout the world, HTLV-1 is highly geographically restricted, being found primarily in southern Japan, the Caribbean, west and central Africa, and the South Pacific islands. Evidence for causality includes the monoclonal integration of viral genome in almost all cases of ATL in carriers. The risk factors for HTLV-1-associated malignancy appear to be perinatal infection, high viral load, and being male sex.

Adult T cell leukemia is a cancer of the blood and bone marrow. The standard treatments for adult T cell leukemia/lymphoma are radiation therapy, immunotherapy, and chemotherapy. Radiation therapy may be external beam radiation therapy or brachytherapy. Other methods of treating adult T cell leukemia/lymphoma include immunotherapy and high-dose chemotherapy with stem cell transplantation.

The methods provided by the invention can provide a beneficial effect for Adult T cell leukemia patients, by administration of a nitrobenzamide compound with a topoisomerase inhibitor, or a combination of administration of a nitrobenzamide compound with a topoisomerase inhibitor and radiation therapy, chemotherapy, immunotherapy and high-dose chemotherapy with stem cell transplantation, or a combination thereof.

Viral-Induced Cervical Cancer

Infection of the cervix with human papillomavirus (HPV) is the most common cause of cervical cancer. Not all women with HPV infection, however, will develop cervical cancer. Cervical cancer usually develops slowly over time. Before cancer appears in the cervix, the cells of the cervix go through changes known as dysplasia, in which cells that are not normal begin to appear in the cervical tissue. Later, cancer cells start to grow and spread more deeply into the cervix and to surrounding areas. The standard treatments for cervical cancers are surgery, immunotherapy, radiation therapy and chemotherapy. The types of surgery that may be used are conization, total hysterectomy, bilateral salpingo-oophorectomy, radical hysterectomy, pelvic exenteration, cryosurgery, laser surgery and loop electrosurgical excision procedure. Radiation therapy may be external beam radiation therapy or brachytherapy.

The methods provided by the invention can provide a beneficial effect for adult cervical cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### CNS Cancers

Brain and spinal cord tumors are abnormal growths of tissue found inside the skull or the bony spinal column, which are the primary components of the central nervous system (CNS). Benign tumors are non-cancerous, and malignant tumors are cancerous. The CNS is housed within rigid, bony quarters (i.e., the skull and spinal column), so any abnormal growth, whether benign or malignant, can place pressure on sensitive tissues and impair function. Tumors that originate in the brain or spinal cord are called primary tumors. Most primary tumors are caused by out-of-control growth among cells that surround and support neurons. In a small number of individuals, primary tumors may result from specific genetic disease (e.g., neurofibromatosis, tuberous sclerosis) or from exposure to radiation or cancer-causing chemicals. The cause of most primary tumors remains a mystery.

The first test to diagnose brain and spinal column tumors is a neurological examination. Special imaging techniques (computed tomography, and magnetic resonance imaging, positron emission tomography) are also employed. Laboratory tests include the EEG and the spinal tap. A biopsy, a surgical procedure in which a sample of tissue is taken from a suspected tumor, helps doctors diagnose the type of tumor.

Tumors are classified according to the kind of cell from which the tumor seems to originate. The most common primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme) and account for 65% of all primary central nervous system tumors. Some of the tumors are, but not limited to, Oligodendroglioma, Ependymoma, Meningioma, Lymphoma, Schwannoma, and Medulloblastoma.

Neuroepithelial Tumors of the CNS

Astrocytic tumors, such as astrocytoma; anaplastic (malignant) astrocytoma, such as hemispheric, diencephalic, optic, brain stem, cerebellar; glioblastoma multiforme; pilocytic astrocytoma, such as hemispheric, diencephalic, optic, brain stem, cerebellar; subependymal giant cell astrocytoma; and pleomorphic xanthoastrocytoma. Oligodendroglial tumors, such as oligodendroglioma; and anaplastic (malignant) oligodendroglioma. Ependymal cell tumors, such as ependymoma; anaplastic ependymoma; myxopapillary ependymoma; and subependymoma. Mixed gliomas, such as mixed oligoastrocytoma; anaplastic (malignant) oligoastrocytoma; and others (e.g. ependymo-astrocytomas). Neuroepithelial tumors of uncertain origin, such as polar spongioblastoma; astroblastoma; and gliomatosis cerebri. Tumors of the choroid plexus, such as choroid plexus papilloma; and choroid plexus carcinoma (anaplastic choroid plexus papilloma). Neuronal and mixed neuronal-glial tumors, such as gangliocytoma; dysplastic gangliocytoma of cerebellum (Lhermitte-Duclos); ganglioglioma; anaplastic (malignant) ganglioglioma; desmoplastic infantile ganglioglioma, such as desmoplastic infantile astrocytoma; central neurocytoma; dysembryoplastic neuroepithelial tumor; olfactory neuroblastoma (esthesioneuroblastoma. Pineal Parenchyma Tumors, such as pineocytoma; pineoblastoma; and mixed pineocytoma/pineoblastoma. Tumors with neuroblastic or glioblastic elements (embryonal tumors), such as medulloepithelioma; primitive neuroectodermal tumors with multipotent differentiation, such as medulloblastoma; cerebral primitive neuroectodermal tumor; neuroblastoma; retinoblastoma; and ependymoblastoma.

Other CNS Neoplasms

Tumors of the Sellar Region, such as pituitary adenoma; pituitary carcinoma; and craniopharyngioma. Hematopoietic tumors, such as primary malignant lymphomas; plasmacytoma; and granulocytic sarcoma. Germ Cell Tumors, such as germinoma; embryonal carcinoma; yolk sac tumor (endodermal sinus tumor); choriocarcinoma; teratoma; and mixed germ cell tumors. Tumors of the Meninges, such as meningioma; atypical meningioma; and anaplastic (malignant) meningioma. Non-menigothelial tumors of the meninges, such as Benign Mesenchymal; Malignant Mesenchymal; Primary Melanocytic Lesions; Hemopoietic Neoplasms; and Tumors of Uncertain Histogenesis, such as hemangioblastoma (capillary hemangioblastoma). Tumors of Cranial and Spinal Nerves, such as schwannoma (neurinoma, neurilemoma); neurofibroma; malignant peripheral nerve sheath tumor (malignant schwannoma), such as epithelioid, divergent mesenchymal or epithelial differentiation, and melanotic. Local Extensions from Regional Tumors; such as paraganglioma (chemodectoma); chordoma; chodroma; chondrosarcoma; and carcinoma. Metastatic tumors, Unclassified Tumors and Cysts and Tumor-like Lesions, such as Rathke cleft cyst; Epidermoid; dermoid; colloid cyst of the third ventricle; enterogenous cyst; neuroglial cyst; granular cell tumor (choristoma, pituicytoma); hypothalamic neuronal hamartoma; nasal glial herterotopia; and plasma cell granuloma.

Chemotherapeutics available are, but not limited to, alkylating agents such as, Cyclophosphamide, Ifosphamide, Melphalan, Chlorambucil, BCNU, CCNU, Decarbazine, Procarbazine, Busulfan, and Thiotepa; antimetabolites such as, Methotraxate, 5-Fluorouracil, Cytarabine, Gemcitabine (Gemzar®), 6-mercaptopurine, 6-thioguanine, Fludarabine, and Cladribine; anthracyclins such as, daunorubicin. Doxorubicin, Idarubicin, Epirubicin and Mitoxantrone; antibiotics such as, Bleomycin; camptothecins such as, irinotecan and topotecan; taxanes such as, paclitaxel and docetaxel; and platinums such as, Cisplatin, carboplatin, and Oxaliplatin.

The treatments are surgery, radiation therapy, immunotherapy, hyperthermia, gene therapy, chemotherapy, and combination of radiation and chemotherapy. Doctors also may prescribe steroids to reduce the swelling inside the CNS.

The methods provided by the invention can provide a beneficial effect for adult cervical cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### PNS Cancers

The peripheral nervous system consists of the nerves that branch out from the brain and spinal cord. These nerves form the communication network between the CNS and the body parts. The peripheral nervous system is further subdivided into the somatic nervous system and the autonomic nervous system. The somatic nervous system consists of nerves that go to the skin and muscles and is involved in conscious activities. The autonomic nervous system consists of nerves that connect the CNS to the visceral organs such as the heart, stomach, and intestines. It mediates unconscious activities.

Acoustic neuromas are benign fibrous growths that arise from the balance nerve, also called the eighth cranial nerve or vestibulocochlear nerve. These tumors are non-malignant, meaning that they do not spread or metastasize to other parts of the body. The location of these tumors is deep inside the skull, adjacent to vital brain centers in the brain stem. As the tumors enlarge, they involve surrounding structures which have to do with vital functions. In the majority of cases, these tumors grow slowly over a period of years.

The malignant peripheral nerve sheath tumor (MPNST) is the malignant counterpart to benign soft tissue tumors such as neurofibromas and schwannomas. It is most common in the deep soft tissue, usually in close proximity of a nerve trunk. The most common sites include the sciatic nerve, brachial plexus, and sarcal plexus. The most common symptom is pain which usually prompts a biopsy. It is a rare, aggressive, and lethal orbital neoplasm that usually arises from sensory branches of the trigeminal nerve in adults. Malignant PNS tumor spreads along nerves to involve the brain, and most patients die within 5 years of clinical diagnosis. The MPNST may be classified into three major categories with epithelioid, mesenchymal or glandular characteristics. Some of the MPNST include but not limited to, Subcutaneous malignant epithelioid schwannoma with cartilaginous differentiation, Glandular malignant schwannoma, Malignant peripheral nerve sheath tumor with perineurial differentiation, Cutaneous epithelioid malignant nerve sheath tumor with rhabdoid features, Superficial epithelioid MPNST, Triton Tumor (MPNST with rhabdomyoblastic differentiation), Schwannoma with rhabdomyoblastic differentiation. Rare MPNST cases contain multiple sarcomatous tissue types, especially osteosarcoma, chondrosarcoma and angiosarcoma. These have sometimes been indistinguishable from the malignant mesenchymoma of soft tissue.

Other types of PNS cancers include but not limited to, malignant fibrous cytoma, malignant fibrous histiocytoma, malignant meningioma, malignant mesothelioma, and malignant mixed Mllerian tumor.

The treatments are surgery, radiation therapy, immunotherapy, chemotherapy, and combination of radiation and chemotherapy.

The methods provided by the invention can provide a beneficial effect for PNS cancers, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Oral Cavity and Oropharyngeal Cancer

Management of patients with central nervous system (CNS) cancers remains a formidable task. Cancers such as, hypopharyngeal cancer, laryngeal cancer, nasopharyngeal cancer, oropharyngeal cancer, and the like, have been treated with surgery, immunotherapy, chemotherapy, combination of chemotherapy and radiation therapy. Etoposide and actinomycin D, two commonly used oncology agents that inhibit topoisomerase II, fail to cross the blood-brain barrier in useful amounts.

The methods provided by the invention can provide a beneficial effect for OralCavity and Oropharyngeal cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Stomach Cancer

Stomach cancer is the result of cell changes in the lining of the stomach. There are three main types of stomach cancers: lymphomas, gastric stromal tumors, and carcinoid tumors. Lymphomas are cancers of the immune system tissue that are sometimes found in the wall of the stomach. Gastric stromal tumors develop from the tissue of the stomach wall. Carcinoid tumors are tumors of hormone-producing cells of the stomach.

The causes of stomach cancer continue to be debated. A combination of heredity and environment (diet, smoking, etc) are all thought to play a part. Common approaches to the treatment include surgery, immunotherapy, chemotherapy, radiation therapy, combination of chemotherapy and radiation therapy or biological therapy.

The methods provided by the invention can provide a beneficial effect for stomach cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Gallbladder Cancer

In another aspect, the invention provides methods to treat gallbladder cancers. In some embodiments, the method comprises administering benzopyrone compounds alone into a subject. In other embodiments, the method comprises administering benzopyrone compounds in combination with one or more anti-tumor agents as listed herein into a subject.

Gallbladder cancer is a rare cancer in which malignant cells are found in the tissues of the gallbladder. The gallbladder stores bile, a fluid made by the liver to digest fat. The wall of the gallbladder has 3 main layers of tissue: mucosal (innermost) layer, muscularis (middle, muscle) layer, and serosal (outer) layer. Between these layers is supporting connective tissue. Primary gallbladder cancer starts in the innermost layer and spreads through the outer layers as it grows. Gallbladder cancer can be cured only if it is found before it has spread, when it can be removed by surgery. If the cancer has spread, palliative treatment can improve the patient's quality of life by controlling the symptoms and complications of this disease.

The methods provided by the invention can provide a beneficial effect for gallbladder cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Esophageal Cancer

In another aspect, the invention provides methods to treat esophageal cancers. In some embodiments, the method comprises administering benzopyrone compounds alone into a subject. In other embodiments, the method comprises administering benzopyrone compounds in combination with one or more anti-tumor agents as listed herein into a subject.

Esophageal cancer is malignancy of the esophagus. There are various subtypes. Most tumors of the esophagus are malignant. A very small proportion (under 10%) is leiomyoma (smooth muscle tumor) or gastrointestinal stromal tumor (GIST). Malignant tumors are generally adenocarcinomas, squamous cell carcinomas, and occasionally small-cell carcinomas. The latter share many properties with small-cell lung cancer, and are relatively sensitive to chemotherapy compared to the other types.

Small and localized tumors are treated surgically with curative intent. Larger tumors tend not to be operable and hence cannot be cured; their growth can still be delayed with chemotherapy, radiotherapy or a combination of the two. In some cases chemo- and radiotherapy can render these larger tumors operable.

The methods provided by the invention can provide a beneficial effect for esophageal cancer patients, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Testicular Cancer

Testicular cancer is cancer that typically develops in one or both testicles in young men. Cancers of the testicle develop in certain cells known as germ cells. The 2 main types of germ cell tumors (GCTs) that occur in men are seminomas (60%) and nonseminomas (40%). Tumors can also arise in the supportive and hormone-producing tissues, or stroma, of the testicles. Such tumors are known as gonadal stromal tumors. The 2 main types are Leydig cell tumors and Sertoli cell tumors. Secondary testicular tumors are those that start in another organ and then spread to the testicle. Lymphoma is the most common secondary testicular cancer.

Common approaches to the treatment include surgery, immunotherapy, chemotherapy, radiation therapy, combination of chemotherapy and radiation therapy or biological therapy. Several drugs are typically used to treat testicular cancer: Platinol (cisplatin), Vepesid or VP-16 (etoposide) and Blenoxane (bleomycin sulfate). Additionally, Ifex (ifosfamide), Velban (vinblastine sulfate) and others may be used.

The methods provided by the invention can provide a beneficial effect for stomach cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Thymus Cancer

The thymus is a small organ located in the upper/front portion of your chest, extending from the base of the throat to the front of the heart. The thymus contains 2 main types of cells, thymic epithelial cells and lymphocytes. Thymic epithelial cells can give origin to thymomas and thymic carcinomas. Lymphocytes, whether in the thymus or in the lymph nodes, can become malignant and develop into cancers called Hodgkin disease and non-Hodgkin lymphomas. The thymus also contains another much less common type of cells called Kulchitsky cells, or neuroendocrine cells, which normally release certain hormones. These cells can give rise to cancers, called carcinoids or carcinoid tumors that often release the same type of hormones, and are similar to other tumors arising from neuroendocrine cells elsewhere in the body.

Common approaches to the treatment include surgery, immunotherapy, chemotherapy, radiation therapy, combination of chemotherapy and radiation therapy or biological therapy. Anticancer drugs that have been used in the treatment of thymomas and thymic carcinomas are doxorubicin (adriamycin), cisplatin, ifosfamide, and corticosteroids (prednisone). Often, these drugs are given in combination to increase their effectiveness. Combinations used to treat thymic cancer include cisplatin, doxorubicin, etoposide and cyclophosphamide, and the combination of cisplatin, doxorubicin, cyclophosphamide, and vincristine.

The methods provided by the invention can provide a beneficial effect for stomach cancer, by administration of a nitrobenzamide compound with a topoisomerase inhibitor, or a combination of administration of a nitrobenzamide compound with a topoisomerase inhibitor and surgery, radiation therapy, chemotherapy, or a combination thereof.

### Combination Therapy

Some embodiments provide combinations of one or more PARP inhibitors described herein and one or more topoisomerase inhibitors described herein. In some embodiments, the PARP inhibitor is 4-iodo-3-nitrobenzamide or a pharmaceutically acceptable salt, pro-drug or metabolite thereof. In some embodiments, the topoisomerase inhibitor is topotecan, irinotecan, lurtotecan, exatecan or a pharmaceutically acceptable salt or metabolite thereof. In some preferred embodiments, the combination is of BA or pharmaceutically acceptable salt or metabolite thereof and topotecan or a pharmaceutically acceptable salt thereof.

In other embodiments of the present invention, the methods of the invention further comprise treating cancer by administering to a subject a PARP inhibitor with at least one topoisomerase inhibitor in combination with another anti-cancer therapy including but not limited to surgery, radiation therapy (e.g. X ray), gene therapy, immunotherapy, DNA therapy, adjuvant therapy, neoadjuvant therapy, viral therapy, RNA therapy, or nanotherapy.

Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time. The conjugate and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

### Radiation Therapy

Radiation therapy (or radiotherapy) is the medical use of ionizing radiation as part of cancer treatment to control malignant cells. Radiotherapy may be used for curative or adjuvant cancer treatment. It is used as palliative treatment (where cure is not possible and the aim is for local disease control or symptomatic relief) or as therapeutic treatment (where the therapy has survival benefit and it can be curative). Radiotherapy is used for the treatment of malignant tumors and may be used as the primary therapy. It is also common to combine radiotherapy with surgery, chemotherapy, hormone therapy or some mixture of the three. Most common cancer types can be treated with radiotherapy in some way. The precise treatment intent (curative, adjuvant, neoadjuvant, therapeutic, or palliative) will depend on the tumour type, location, and stage, as well as the general health of the patient.

Radiation therapy is commonly applied to the cancerous tumor. The radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion.

Radiation therapy works by damaging the DNA of cells. The damage is caused by a photon, electron, proton, neutron, or ion beam directly or indirectly ionizing the atoms which make up the DNA chain. Indirect ionization happens as a result of the ionization of water, forming free radicals, notably hydroxyl radicals, which then damage the DNA. In the most common forms of radiation therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Proton radiotherapy works by sending protons with varying kinetic energy to precisely stop at the tumor.

Gamma rays are also used to treat certain types of cancer. In the procedure called gamma-knife surgery, multiple concentrated beams of gamma rays are directed on the growth in order to kill the cancerous cells. The beams are aimed from different angles to focus the radiation on the growth while minimizing damage to the surrounding tissues.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the patient directly, or ex vivo, with infected cells being introduced back into a patient. Such compositions are suitable for use in the present invention.

### Adjuvant Therapy

Adjuvant therapy is a treatment given after the primary treatment to increase the chances of a cure. Adjuvant therapy may include chemotherapy, radiation therapy, hormone therapy, or biological therapy. Which adjuvant therapy is best for a patient is based on the type of cancer and its stage.

Because the principal purpose of adjuvant therapy is to kill any cancer cells that may have spread, treatment is usually systemic (uses substances that travel through the bloodstream, reaching and affecting cancer cells all over the body).

Adjuvant chemotherapy is the use of drugs to kill cancer cells. Chemotherapy can reach nearly every part of the body to kill cancer cells. Adjuvant chemotherapy is usually a combination of anticancer drugs, which has been shown to be more effective than a single anticancer drug.

Some cancers are sensitive to hormones. By reducing hormone production or by blocking the cancer's ability to accept the hormones, hormone therapy can prevent cancer cells from growing. Hormone therapy can be used in conjunction with surgery, radiation or chemotherapy.

Radiation therapy is sometimes used as a local adjuvant treatment. Radiation therapy is considered adjuvant treatment when it is given before or after a mastectomy.

### Neoadjuvant Therapy

Neoadjuvant therapy refers to a treatment given before the primary treatment. Examples of neoadjuvant therapy include chemotherapy, radiation therapy, and hormone therapy.

### Oncolytic Viral Therapy

Viral therapy for cancer utilizes a type of viruses called oncolytic viruses. An oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site.

There are two main approaches for generating tumor selectivity: transductional and non-transductional targeting. Transductional targeting involves modifying the specificity of viral coat protein, thus increasing entry into target cells while reducing entry to non-target cells. Non-transductional targeting involves altering the genome of the virus so it can only replicate in cancer cells. This can be done by either transcription targeting, where genes essential for viral replication are placed under the control of a tumor-specific promoter, or by attenuation, which involves introducing deletions into the viral genome that eliminate functions that are dispensable in cancer cells, but not in normal cells. There are also other, slightly more obscure methods.

Chen et al (2001) used CV706, a prostate-specific adenovirus, in conjunction with radiotherapy on prostate cancer in mice. The combined treatment results in a synergistic increase in cell death, as well as a significant increase in viral burst size (the number of virus particles released from each cell lysis).

ONYX-015 has undergone trials in conjunction with chemotherapy. The combined treatment gives a greater response than either treatment alone, but the results have not been entirely conclusive. ONYX-015 has shown promise in conjunction with radiotherapy.

Viral agents administered intravenously can be particularly effective against metastatic cancers, which are especially difficult to treat conventionally. However, bloodborne viruses can be deactivated by antibodies and cleared from the blood stream quickly e.g. by Kupffer cells (extremely active phagocytic cells in the liver, which are responsible for adenovirus clearance). Avoidance of the immune system until the tumour is destroyed could be the biggest obstacle to the success of oncolytic virus therapy. To date, no technique used to evade the immune system is entirely satisfactory. It is in conjunction with conventional cancer therapies that oncolytic viruses show the most promise, since combined therapies operate synergistically with no apparent negative effects.

The specificity and flexibility of oncolytic viruses means they have the potential to treat a wide range of cancers with minimal side effects. Oncolytic viruses have the potential to solve the problem of selectively killing cancer cells.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formuation and manufacturing process for cancer nanotherapy is disclosed in U.S. Pat. No. 7,179,484, and article M. N. Khalid, P. Simard, D. Hoarau, A. Dragomir, J. Leroux, Long Circulating Poly(Ethylene Glycol)Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to down-regulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Cand5, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Administration with such chemopreventative agents in combination with eflomithine-NSAID conjugates of the invention can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylomithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

### Formulations, Routes of Administration, and Effective Doses

Another aspect of the present invention relates to formulations and routes of administration for pharmaceutical compositions comprising a nitrobenzamide compound. Such pharmaceutical compositions can be used to treat cancer in the methods described in detail above.

The compounds of formula Ia may be provided as a prodrug and/or may be allowed to interconvert to a nitrosobenzamide form in vivo after administration. That is, either the nitrobenzamide form and/or the nitrosobenzamide form, or pharmaceutically acceptable salts may be used in developing a formulation for use in the present invention. Further, in some embodiments, the compound may be used in combination with one or more other compounds or in one or more other forms. For example a formulation may comprise both the nitrobenzamide compound and acid forms in particular proportions, depending on the relative potencies of each and the intended indication. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

In compositions comprising combinations of a nitrobenzamide compound and another active agent can be effective. The two compounds and/or forms of a compound may be formulated together, in the same dosage unit e.g. in one cream, suppository, tablet, capsule, or packet of powder to be dissolved in a beverage; or each form may be formulated in separate units, e.g., two creams, suppositories, tablets, two capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of the compound of the invention in treating a cancer.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions may contain a nitrobenzamide compound with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol, 23: 238-252 (1965) and Szoka et al., Proc. Natl. Acad. Sci. 75: 4194-4198 (1978), incorporated herein by reference. Ligands may also be attached to the liposomes to direct these compositions to particular sites of action. Compounds of this invention may also be integrated into foodstuffs, e.g, cream cheese, butter, salad dressing, or ice cream to facilitate solubilization, administration, and/or compliance in certain patient populations.

Pharmaceutical preparations for oral use may be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

For injection, the inhibitors of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for use in the present invention include compositions wherein the active ingredients are present in an effective amount, i.e., in an amount effective to achieve therapeutic and/or prophylactic benefit in at least one of the cancers described herein. The actual amount effective for a particular application will depend on the condition or conditions being treated, the condition of the subject, the formulation, and the route of administration, as well as other factors known to those of skill in the art. Determination of an effective amount of a nitrobenzamide compound is well within the capabilities of those skilled in the art, in light of the disclosure herein, and will be determined using routine optimization techniques.

In some embodiments, in order to carry out the current invention, the compositions and methods disclosed in other patents and patent applications assigned to BiPar are used. For example, formulations for treating cancer as described in U.S. patent application Ser. No. 12/015,403 and PCT Application PCT/US2008/51214 can be used. All patents and patent applications are herein incorporated by reference in their entirety.

### EXAMPLES

### Example 1

### In Vitro Combination Effects of 4-iodo-3-nitrobenzamide (BA) with Irinotecan

The in vitro combination effects of 4-Iodo-3-nitrobenzamide (BA) with irinotecan hydrochloride are examined using human small cell lung cancer cells, LX-1 cells.

### Cell Culture

Human small cell lung cancer cell strain LX-1 is obtained from ATCC (American Type Culture Collection). In a medium comprising D-MEM (Dulbecco's Modified Eagle Medium) and 10% bovine fetal serum (FCS), human small cell lung cancer cell strain LX-1 is subcultured. The culture is carried out in an incubator with 5% CO₂ at 37° C. The same medium is also used in the following experiments. LX-1 cells on subculture are subjected to a trypsin treatment, suspended in the medium and plated at 10⁵ cells per P100 cell culture dish or at 10⁴ cells per P60 cell culture dish in the presence of different concentrations compounds or DMSO control. Following treatment, the number of attached cells is measured using Coulter counter, and by staining with 1% methylene blue. Methylene blue is dissolved in 50%-50% mixture of Methanol and water. Cells are plated in 24- or 96-well plates and treated as planned, media are aspirated, cells are washed with PBS, fixed in methanol for 5-10 min, methanol is aspirated and plates are allowed to dry completely. Methylene blue solution is added to wells and plates are incubated for 5 min. Staining solution is removed and plates are washed with dH₂O until washes are no longer blue. After plates are completely dry, a small amount of 1N HCl is added to each well to extract the methylene blue. The OD readout at 600 nm and a calibration curve are used to determine cell number.

### Compounds

Test agents are prepared as the following. Irinotecan hydrochloride is obtained from Daiichi Pharmaceutical Co., Ltd. and provided after 2 fold serial dilutions with medium in use. Benzamide compounds are dissolved directly from dry powder to 10 mM stock solution in DMSO for each separate experiment. Control experiments are carried out with the matching volume/concentration of the vehicle (DMSO); in these controls, the cells show no changes in their growth or cell cycle distribution.

### PI Exclusion, Cell Cycle and TUNEL Assays

After the addition of drugs and incubation, cells are trypsinized and aliquots of the samples are taken for counting and PI (Propidium Iodide) exclusion assay. One part of the cells is centrifuged and resuspended in 0.5 ml ice-cold PBS containing 5 μg/ml of PI. The other part of the cells is fixed in ice-cold 70% ethanol and stored in a freezer overnight. For cell cycle analysis, cells are stained with propidium iodide (PI) by standard procedures. Cellular DNA content is determined by flow cytometry using BD LSRII FACS, and the percentages of cells in G1, S or G2/M are determined using ModFit software.

The cells are labeled for apoptosis with the “In Situ Cell Death Detection Kit, Fluorescein” (Roche Diagnostics Corporation, Roche Applied Science, Indianapolis, Ind.). Briefly, fixed cells are centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 min at room temperature and washed twice in 0.2 ml PBS/1% BSA. The cells are resuspended in 50 μl TUNEL reaction mixture (TdT enzyme and labeling solution) and incubated for 60 min at 37° C. in a humidified dark atmosphere in an incubator. The labeled cells are washed once in PBS/1% BSA, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.).

### Bromodeoxyuridine (BrdU) Labeling Assay

50 μl of BrdU (Sigma Chemical Co., St. Louis, Mo.) stock solution (1 mM) is added to give 10 μM BrdU final concentration. The cells are incubated for 30 min at 37° C. and fixed in ice-cold 70% ethanol and stored in a cold room (4° C.) overnight. Fixed cells are centrifuged and washed once in 2 ml PBS, then resuspended in 0.7 ml of denaturation solution (0.2 mg/ml pepsin in 2 N HCl) for 15 min at 37° C. in the dark and suspended with 1.04 ml 1M Tris buffer (Trizma base, Sigma Chemical Co.) and washed in 2 ml PBS. Then cells are resuspended in 100-11 anti-BrdU antibody (DakoCytomation, Carpinteria, Calif.) with 1:100 dilution in TBFP permeable buffer (0.5% Tween-20, 1% bovine serum albumin and 1% fetal bovine serum in PBS) and incubated for 25 min at room temperature in the dark and washed in 2 ml PBS. The primary antibody-labeled cells are resuspended in 100 μl Alexa Fluor F(ab′)2 fragment of goat anti-mouse IgG (H+L) (2 mg/mL) (Molecular Probes, Eugene, Oreg.) with 1:200 dilution in TBFP permeable buffer and incubated for 25 min at room temperature in the dark and washed in 2 ml PBS, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.).

Combinations of 4-iodo-3-nitrobenzamide (BA) with topoisomerase inhibitors, i.e. irinotecan or topotecan, have been tested in in vitro and in vivo models of cancer. Evaluation of BA in combination with irinotecan in the LX-1 small cell lung carcinoma cell line shows that BA potentiates S- and G2/M cell cycle arrest and enhances cytotoxic effects induced by irinotecan.

### Example 2

### In Vivo Anti-Tumor Activity of a Combination of 4-iodo-3-nitrobenzamide (BA) and Irinotecan in the Treatment of Colorectal Cancer

Three colorectal cancer cell lines: CACO-2, HT-29, and DHD/K12/TRb (PROb), are subcutaneously transplanted to nude mice (59 animals) at 6 weeks of age, respectively. After 11 days from the tumor transplantation, 36 animals having a tumor volume of about 100 to 300 mm³ are allotted to 5 groups consisting of 6 animals per group. On the same day, the animals receive parenteral administration, respectively, of cysteine buffer for “vehicle group”, 50 mg/kg or 15 mg/kg of BA (i.p.) biweekly for “BA alone administration group”, 50 mg/kg or 15 mg/kg of irinotecan (i.p.) for “irinotecan alone administration group”, 50 mg/kg of BA (i.p.) and 50 mg/kg of irinotecan (i.p.) for “combined administration group (higher doses), 15 mg/kg of BA (i.p.) and 15 mg/kg of irinotecan (i.p.) for “combined administration group (lower doses)”. Thereafter, tumor volume and body weight of the mice are measured for 30 days.

BA is dissolved directly from dry powder to 10 mM stock solution in DMSO for each separate experiment. Control experiments are carried out with the matching volume/concentration of the vehicle (DMSO). Irinotecan is administered by giving 50 mg/kg or 15 mg/kg of irinotecan intraperitoneally (i.p.).

Endpoint

Tumors are calipered twice weekly for the duration of the study. Each animal is euthanized when its neoplasm reaches the predetermined endpoint size (1,000 mm³). The time to endpoint (TTE) for each mouse is calculated by the following equation:

$$T T E = \frac{{log}_{16} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are euthanized at the end of the study, and assigned a TTE value equal to the last day (68 days). Treatment efficacy is determined from tumor growth delay (TGD), which is defined as the increase in the median TTE for a treatment group compared to the control group:

TGD=T−C,

expressed in days, or as a percentage of the median TTE of the control group:

$$\% T G D = \frac{T - C}{C} \times 100$$

where:
T=median TTE for a treatment group,
C=median TTE for control Group.

MTV and Criteria for Regression Responses

Treatment efficacy is also determined from the tumor volumes of animals remaining in the study on the last day, and from the number of regression responses. The MTV(n) is defined as the median tumor volume on D61 in the number of animals remaining, n, whose tumors have not attained the endpoint volume. Treatment may cause a partial regression (PR) or a complete regression (CR) of the tumor in an animal. A PR indicates that the tumor volume is 50% or less of its D1 volume for three consecutive measurements during the course of the study, and equal to or greater than 13.5 mm³ for one or more of these three measurements. A CR indicates that the tumor volume is less than 13.5 mm³ for three consecutive measurements during the course of the study. An animal with a CR at the termination of a study is additionally classified as a tumor-free survivor (TFS).

Statistical and Graphical Analyses

The logrank test is employed to analyze the significance of the difference between the TTE values of two groups by comparing their Kaplan-Meier curves. The logrank test analyzes the data for all animals in a group, except the NTR deaths. The two-tailed statistical analyses are conducted at P=0.05, using Prism 3.03 (GraphPad) for Windows. Prism reports logrank test results as not significant at P>0.05, significant at 0.01<P≦0.05, very significant at 0.001<P≦0.01 and extremely significant at P≦0.001. Because the logrank test determines statistical significance, and does not provide an estimate of the magnitude of the difference between groups, all levels of significance are reported as either significant or non-significant within the text of this report.

Results:

The tumor growth curves show the group median tumor volume as a function of time. The combination of the various doses of BA with irinotecan results in greatly reduced tumor volume as compared to treatment with irinotecan alone.

When an animal exits the study due to tumor size or TR death, the final tumor volume recorded for the animal is included with the data used to calculate the median volume at subsequent time points. Therefore, the final median tumor volume shown by the curve may differ from the MTV, which is the median tumor volume for mice remaining in the study on the last day (excluding all with tumors that have attained the endpoint). If more than one TR death occurs in a group, the median tumor growth curve is truncated at the time of the last measurement that precedes the second TR death. Tumor growth curves are also truncated when the tumors in more than 50% of the assessable animals in a group have attained the endpoint volume.

### Example 3

### Anti-Tumor Effect of a Combination of 4-iodo-3-nitrobenzamide (BA) and Topotecan in Treating Small Cell Lung Cancer

The anti-tumor effect of a combination of BA and topotecan, one of the approved drugs for the treatment of small cell lung cancer (SCLC) in humans, is evaluated in an established subcutaneous xenograft model of SCLC. SCID mice (24 animals) are inoculated with human small cell lung cancer SW-2 cells (8×10⁶ cells/animal) injected subcutaneously into the right flank of the mice. When the tumors reach about 80 mm³ in size, the mice are randomly divided into four groups (6 animals per group). The first group of mice is treated with topotecan administered i.p. This group of mice is further divided into 3 subgroups, which receive 0.5 mg/kg, 1 mg/kg, or 2 mg/kg of topotecan i.p. respectively. A second group of animals is treated with 4-iodo-3-nitrobenzamide (BA). BA is administrated as a continuous infusion (i.v.) (CI) via Alzet® osmotic pumps (Model 1002), which delivers a total volume of approximately 100 mL, at 0.25 μL/hour for 14 days. Each pump delivers a total dose of 25 mg/kg/week of BA over 14 days. Alzet model osmotic pumps are implanted on days 1, 15, and 29. The pumps are pre-warmed for ˜1 hour at 37° C., and then implanted subcutaneously (s.c.) in the left flanks of isofluoraneanesthetized mice. The third group of mice receives a combination of topotecan and BA, using the same doses and schedules as in groups 1 and 2. A control group of animals receives phosphate-buffered saline (PBS) using the same schedule as the animals in group 2. Tumor growth is monitored by measuring tumor size twice per week. Tumor size is calculated using the formula: length×width×height×(½).

Change in tumor size is monitored twice weekly and then daily. In the control group of animals, tumors grow to about 800³ mm in 44 days. Treatment with topotecan alone results in tumor growth delays of 12 days. Treatment with BA alone results in a tumor-growth delay of 34 days in 3 out of 6 animals. The remaining 3 animals in this group have complete tumor regressions. Treatment with the combination of topotecan and BA shows an enhanced anti-tumor effect resulting in complete tumor regression in 5 out of the 6 treated animals. These animals are tumor-free on day 78, the last measurement point. Thus, the combination of topotecan and BA is synergistic when compared to the single agents in this human SCLC xenograft model.

### Example 4

### Anti-Tumor Effect of a Combination of 4-iodo-3-nitrobenzamide (BA) and Topotecan in Treating Cervical Cancer

Immunodeficient, SCID, mice receive topotecan at two dose levels as monotherapies, and in combinations with 4-iodo-3-nitrobenzamide (BA), which is administered via three sequential 14-day infusions. Treatments begin on Day 1 (D1), and animals are euthanized when their tumors attained the 750 mm³ endpoint volume.

The study examines the effects of continuous BA infusions on topotecan activity and tolerability in SCID mice bearing established SiHa carcinomas.

Mice

Female CB.17 SCID mice (Charles River) are 10 weeks old, and have a body weight (BW) range of 15.2-26.6 g on D1 of the study. The animals are fed ad libitum water (reverse osmosis, 1 ppm Cl) and NIH 31 Modified and Irradiated Lab Diet® consisting of 18.0% crude protein, 5.0% crude fat, and 5.0% crude fiber. The mice are housed on irradiated ALPHA-dri® bed-o-cobs® Laboratory Animal Bedding in static microisolators on a 12-hour light cycle at 21-22° C. (70-72° F.) and 40-60% humidity in the laboratory accredited by AAALAC International (Association for Assessment and Accreditation of Laboratory), which assures compliance with accepted standards for the care and use of laboratory animals.

Tumor Implantation

The human SiHa cells, derived from a surgically removed cervical carcinoma, are maintained in athymic nude mice by serial engraftment. A tumor fragment (1 mm³) is implanted s.c. into the right flank of each test mouse. Tumors are monitored twice weekly and then daily as their volumes approach 80-120 mm³. On D1 of the study, animals are sorted into treatment groups with tumor sizes of 63-144 mm³ and group mean tumor sizes of ˜102 mm³.
Tumor size, in mm³, was calculated from:

$$Tumor Volume = \frac{{w}^{2} \times 1}{2}$$

Tumor weight may be estimated with the assumption that 1 mg is equivalent to 1 mm³ of tumor volume.

Treatment

Mice are sorted into groups (n=10) and treated in accordance with the protocol.

4-iodo-3-nitrobenzamide (BA) is administrated intraperitoneally (i.p.) at 15 mg/kg or 50 mg/kg, biweekly. Control Group 1 mice receive the vehicle. Topotecan is administrated intravenously (i.v.), at 0.5 and 1 mg/kg, respectively, once daily on days 1-5, 8-12, and 15-19 (qd×5/2/5/2/5). Starting on day 16, topotecan is administered intraperitoneally (i.p.) at 0.5 mg/kg, 1 mg/kg, or 2 mg/kg.

Endpoint

Tumors are calipered twice weekly for the duration of the study. Each animal is euthanized when its neoplasm reaches the predetermined endpoint size (1,000 mm³). The time to endpoint (TTE) for each mouse is calculated by the following equation:

$$T T E = \frac{{log}_{16} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are euthanized at the end of the study, and assigned a TTE value equal to the last day (68 days). Treatment efficacy is determined from tumor growth delay (TGD), which is defined as the increase in the median TTE for a treatment group compared to the control group:

TGD=T−C,

expressed in days, or as a percentage of the median TTE of the control group:

$$\% T G D = \frac{T - C}{C} \times 100$$

where:
T=median TTE for a treatment group,
C=median TTE for control Group.

MTV and Criteria for Regression Responses

Treatment efficacy is also determined from the tumor volumes of animals remaining in the study on the last day, and from the number of regression responses. The MTV(n) is defined as the median tumor volume on D61 in the number of animals remaining, n, whose tumors have not attained the endpoint volume. Treatment may cause a partial regression (PR) or a complete regression (CR) of the tumor in an animal. A PR indicates that the tumor volume is 50% or less of its D1 volume for three consecutive measurements during the course of the study, and equal to or greater than 13.5 mm³ for one or more of these three measurements. A CR indicates that the tumor volume is less than 13.5 mm for three consecutive measurements during the course of the study. An animal with a CR at the termination of a study is additionally classified as a tumor-free survivor (TFS).

Statistical and Graphical Analyses

The logrank test is employed to analyze the significance of the difference between the TTE values of two groups by comparing their Kaplan-Meier curves (FIG. 1). The logrank test analyzes the data for all animals in a group, except the NTR deaths. The two-tailed statistical analyses are conducted at P=0.05, using Prism 3.03 (GraphPad) for Windows. Prism reports logrank test results as not significant at P>0.05, significant at 0.01<P≦0.05, very significant at 0.001<P≦0.01 and extremely significant at P≦0.001. Because the logrank test determines statistical significance, and does not provide an estimate of the magnitude of the difference between groups, all levels of significance are reported as either significant or non-significant within the text of this report. The tumor growth curves show the group median tumor volume as a function of time. The combination of BA with topotecan results in greatly reduced tumor volume as compared to treatment with topotecan alone.

When an animal exits the study due to tumor size or TR death, the final tumor volume recorded for the animal is included with the data used to calculate the median volume at subsequent time points. Therefore, the final median tumor volume shown by the curve may differ from the MTV, which is the median tumor volume for mice remaining in the study on the last day (excluding all with tumors that have attained the endpoint). If more than one TR death occurs in a group, the median tumor growth curve is truncated at the time of the last measurement that precedes the second TR death. Tumor growth curves are also truncated when the tumors in more than 50% of the assessable animals in a group have attained the endpoint volume.

The above examples are in no way intended to limit the scope of the instant invention. Further, it can be appreciated to one of ordinary skill in the art that many changes and modifications can be made thereto without departing from the spirit or scope of the appended claims, and such changes and modifications are contemplated within the scope of the instant invention.

It will be apparent to one of ordinary skill in the art that many changes and modifications can be made thereto without departing from the spirit or scope of the appended claims.

What is claimed is:

1. A method of treating a cancer, comprising administering to a patient an effective amount of a combination of a topoisomerase inhibitor and a PARP inhibitor of formula (Ia) 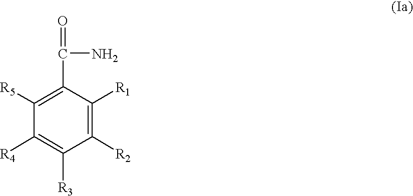
wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof
wherein the cancer is not breast cancer, uterine cancer, or ovarian cancer.

2. The method of claim 1, wherein the PARP inhibitor is of formula:

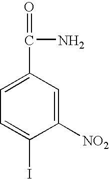

3. The method of claim 1, wherein the PARP inhibitor is a metabolite of 4-iodo-3-nitrobenzamide selected from the group consisting of:

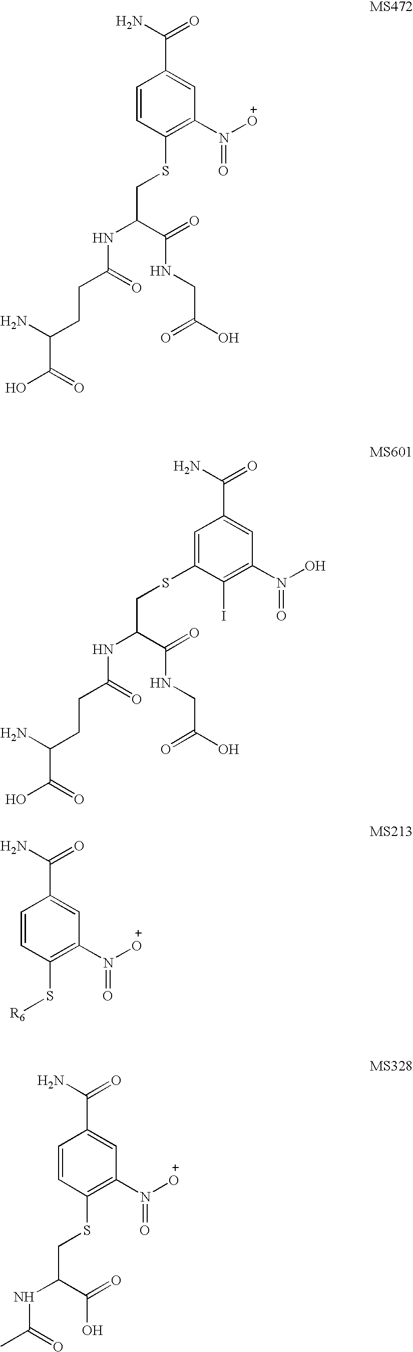

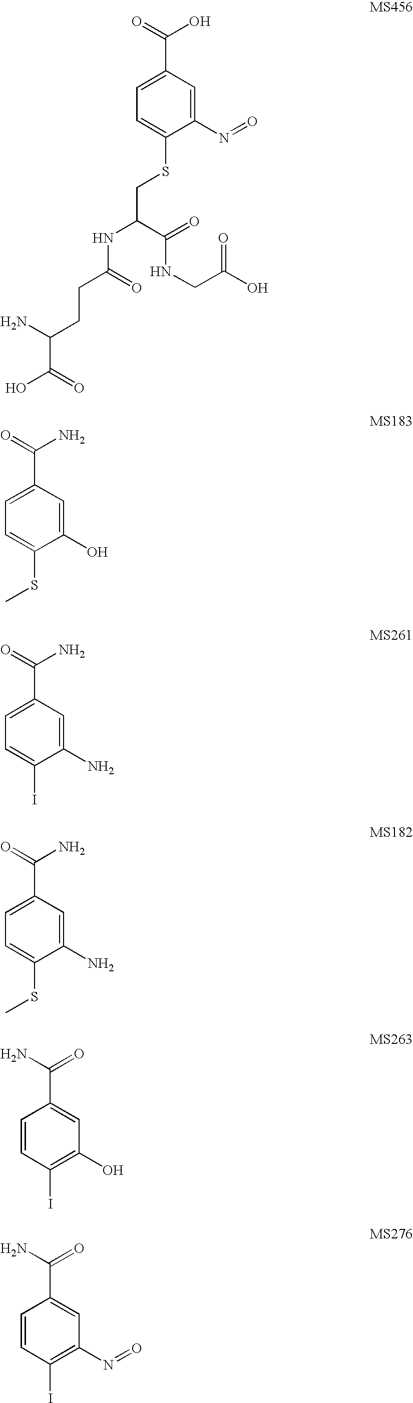

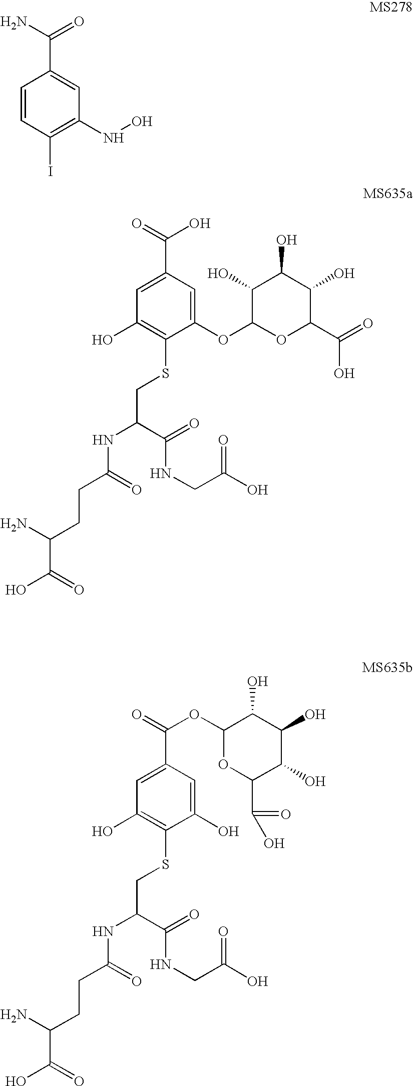

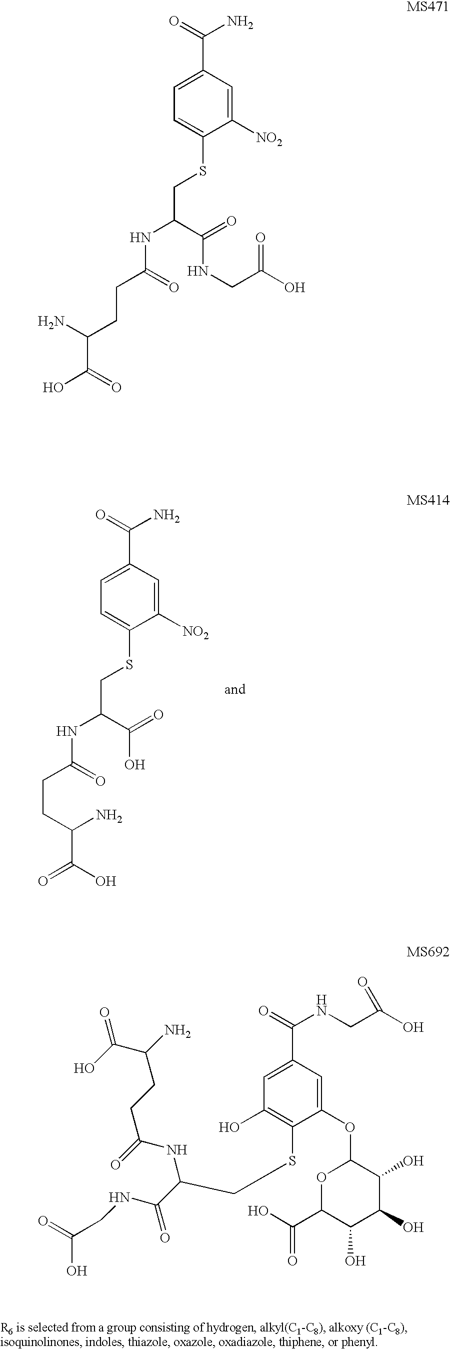

4. The method of claim 1, wherein the topoisomerase inhibitor is topotecan, irinotecan, lurtotecan, exatecan or a pharmaceutically acceptable salt or metabolite thereof.

5. The method of claim 1, wherein the topoisomerase inhibitor is topotecan or a pharmaceutically acceptable salt or metabolite thereof.

6. The method of claim 1, wherein the cancer is selected from adrenal cortical cancer, anal cancer, aplastic anemia, bile duct cancer, bladder cancer, bone cancer, bone metastasis, CNS tumors, peripheral CNS cancer, Castleman's Disease, cervical cancer, childhood Non-Hodgkin's lymphoma, colon and rectum cancer, esophagus cancer, Ewing's family of tumors, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, vaginal cancer, vulvar cancer, Waldenstrom's macroglobulinemia and cancers of viral origin.

7. The method of claim 1, wherein the cancer is selected from the group consisting of leukemia, prostate cancer, transitional cell carcinoma of the bladder, pancreatic cancer, colorectal cancer, cervical cancer, and lung cancer.

8. The method of claim 1, further comprising administering an effective amount of a benzopyrone compound of formula (II): 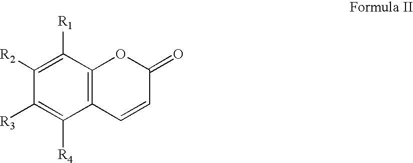
wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

9. The method of claim 1, wherein at least one therapeutic effect is obtained, said at least one therapeutic effect being reduction in size of a tumor, reduction in metastasis, complete remission, partial remission, pathologic complete response, or stable disease.

10. The method of claim 1, wherein an improvement of clinical benefit rate (CBR=CR+PR+SD≧6 months) is obtained as compared to treatment with the topoisomerase inhibitor but without the PARP inhibitor.

11. The method of claim 10, wherein the improvement of clinical benefit rate is at least about 60%.

12. The method of claim 1 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

13. The method of claim 1, wherein the topoisomerase inhibitor is administered as an intravenous infusion.

14. The method of claim 1, wherein 4-iodo-3-nitrobenzamide or its metabolite is administered orally or as a parenteral injection or infusion, or inhalation.

15. The method of claim 1, wherein the PARP inhibitor is administered prior to, or concurrently with, or subsequent to the administration of the topoisomerase inhibitor.

16. The method of claim 1, wherein the PARP inhibitor and the topoisomerase inhibitor are administered in the same formulation.

17. The method of claim 1, wherein the PARP inhibitor and the topoisomerase inhibitor are administered in different formulations.

18. A composition for administration to a patient for the treatment of cancer, the composition comprising an effective amount of a combination of a topoisomerase inhibitor and a PARP inhibitor of formula (Ia): 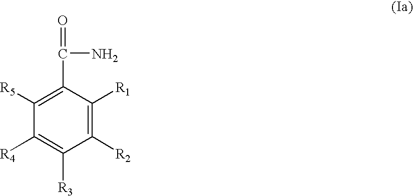
wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs or prodrugs thereof;
wherein the cancer is not breast cancer, uterine cancer, or ovarian cancer.

19. The composition of claim 18, wherein the PARP inhibitor is of formula:

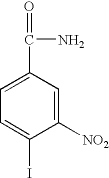

20. The composition of claim 18, wherein the PARP inhibitor is a metabolite of 4-iodo-3-nitrobenzamide selected from the group consisting of:

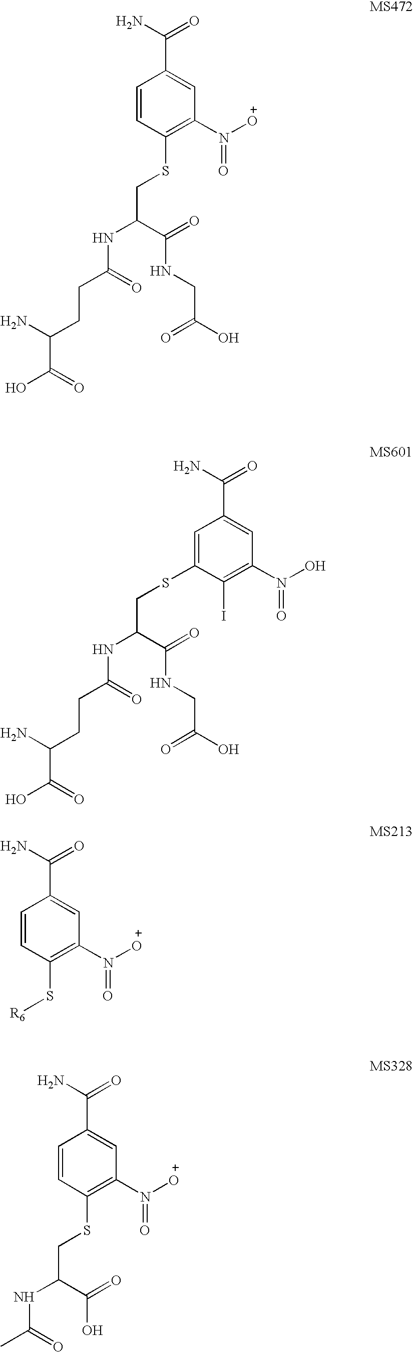

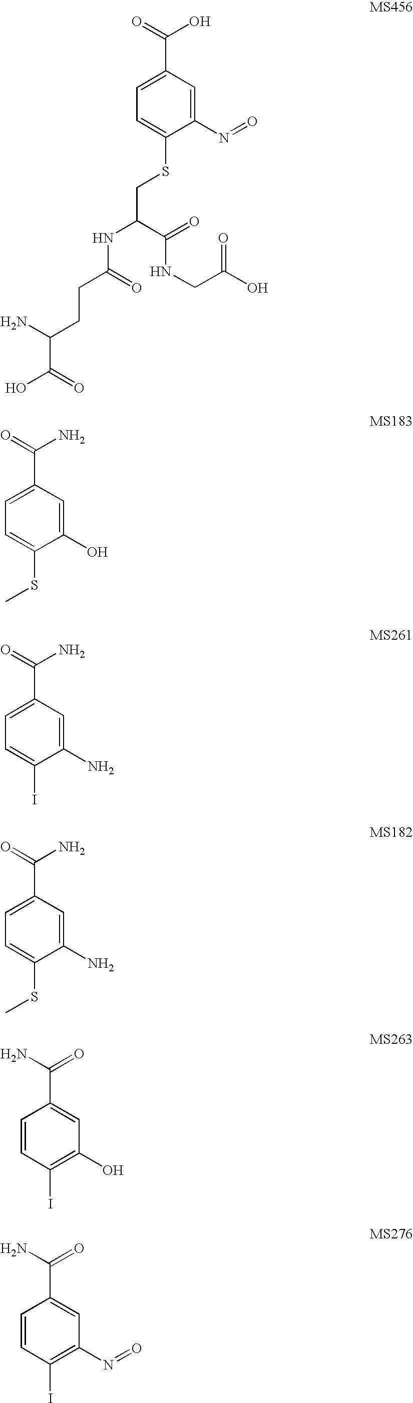

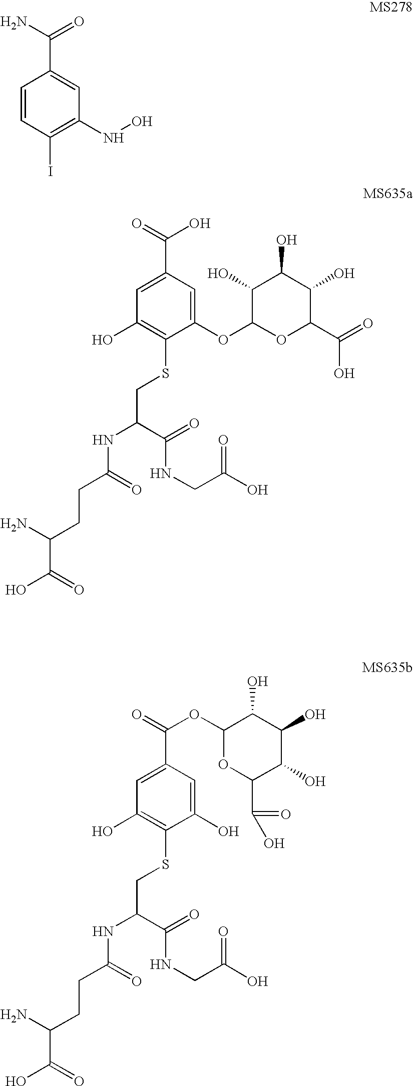

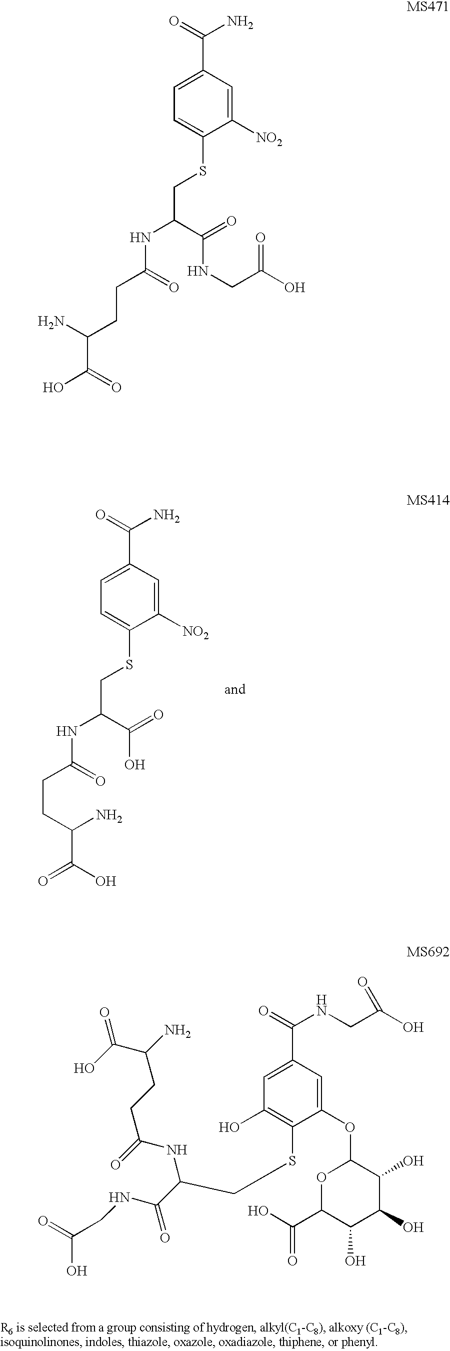

21. The composition of claim 18, wherein the topoisomerase inhibitor is topotecan, irinotecan, lurtotecan, exatecan or a pharmaceutically acceptable salt or metabolite thereof.

22. The composition of claim 18, wherein the topoisomerase inhibitor is topotecan or a pharmaceutically acceptable salt or metabolite thereof.

23. The composition of claim 18, wherein the cancer is selected from the group consisting of leukemia, prostate cancer, transitional cell carcinoma of the bladder, pancreatic cancer, colorectal cancer, cervical cancer, and lung cancer.

24. The composition of claim 18 further comprises an effective amount of a benzopyrone compound of formula (II): 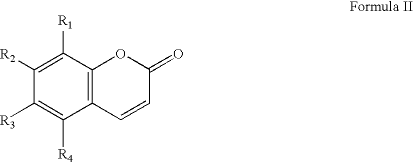
wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

25. The composition of claim 18, wherein the composition is administered in unit dosage form.

26. The composition of claim 25, wherein the unit dosage form is adapted for oral or parenteral administration.

27. The composition of claim 18, wherein upon administration of the composition, at least one therapeutic effect is obtained, said at least one therapeutic effect being reduction in size of a tumor, reduction in metastasis, complete remission, partial remission, pathologic complete response, or stable disease.

28. The composition of claim 18, wherein upon administration of the composition, an improvement of clinical benefit rate (CBR=CR+PR+SD≧6 months) is obtained as compared to treatment with the topoisomerase inhibitor but without the PARP inhibitor.

29. The composition of claim 28, wherein the improvement of clinical benefit rate is at least about 60%.

30. The composition of claim 18, wherein the composition is administered in combination with surgery, radiation therapy, chemotherapy, gene therapy, DNA therapy, adjuvant therapy, neoadjuvant therapy, viral therapy, RNA therapy, immunotherapy, nanotherapy or a combination thereof.

31. A kit for treatment of cancer, comprising:
(a) a PARP inhibitor of the formula (Ia): 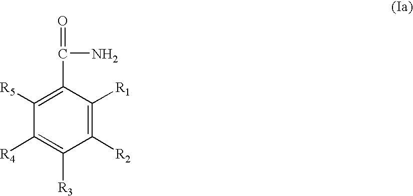
wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs or prodrugs thereof; and
(b) a topoisomerase inhibitor;
wherein the cancer is not breast cancer, uterine cancer, or ovarian cancer.

32. The kit of claim 31, wherein the PARP inhibitor is of formula:

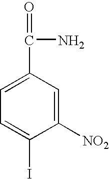

33. The kit of claim 31, wherein the PARP inhibitor is a metabolite of 4-iodo-3-nitrobenzamide selected from the group consisting of:

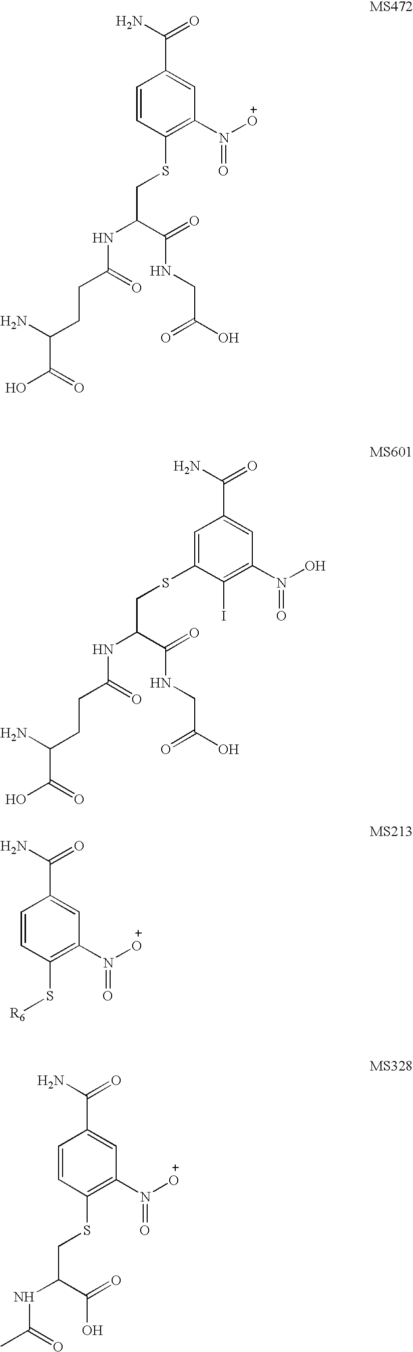

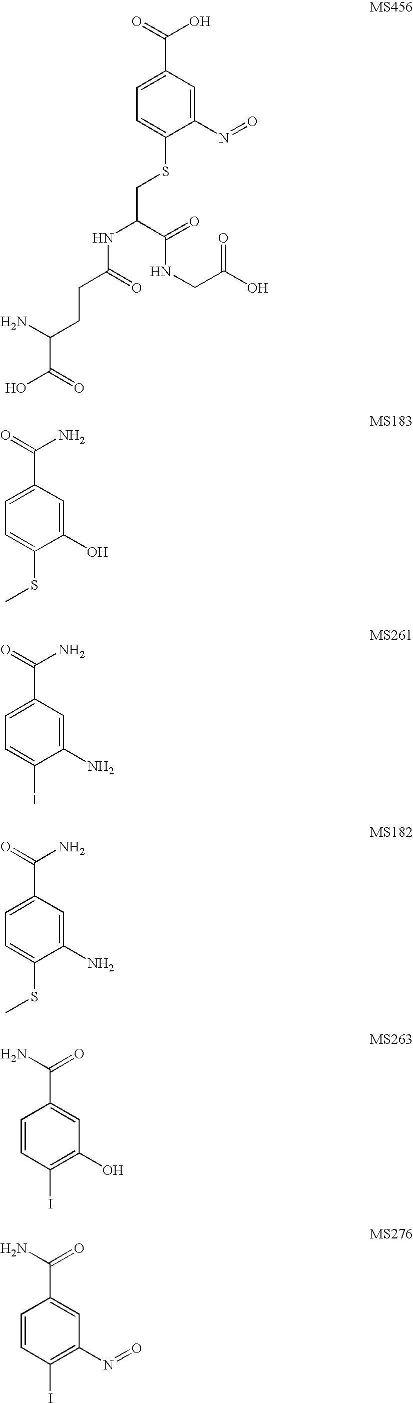

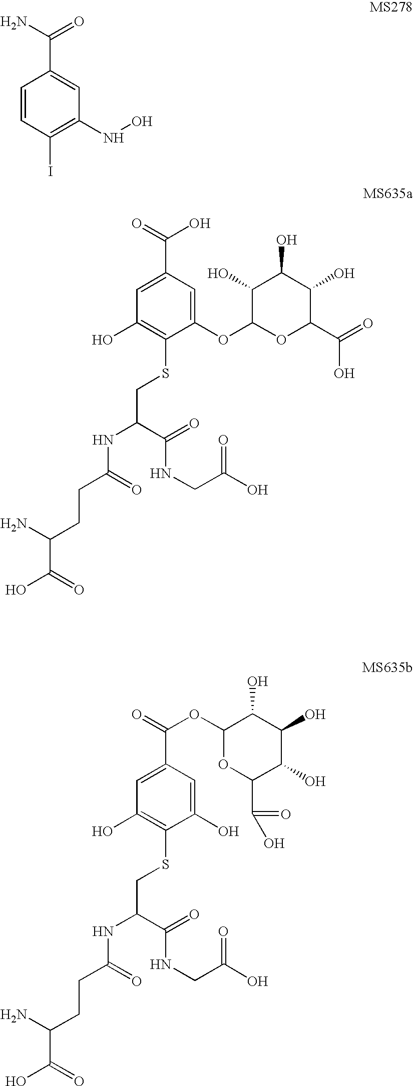

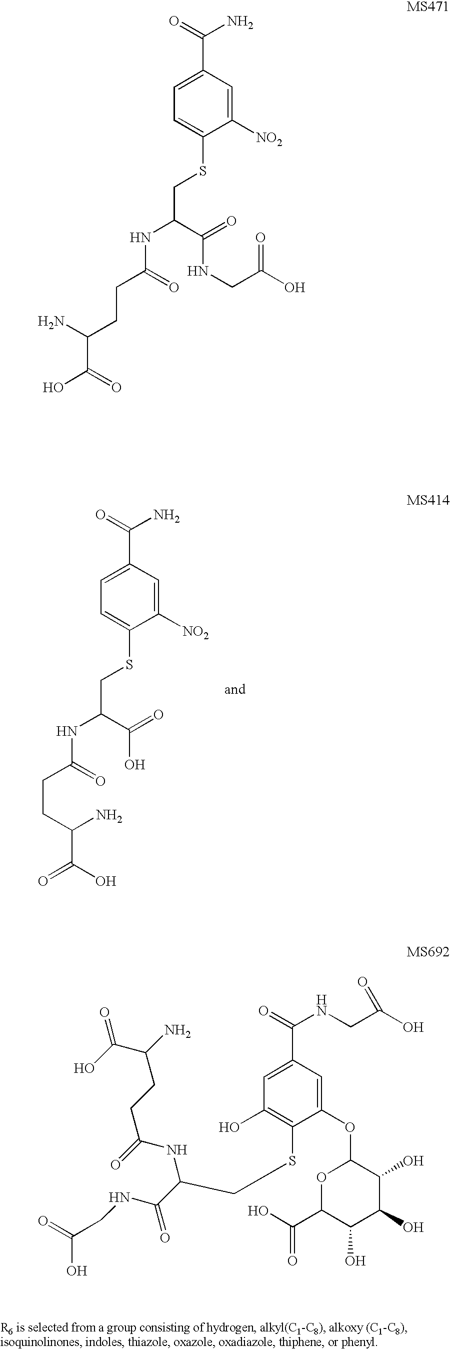

34. The kit of claim 31, wherein the topoisomerase inhibitor is topotecan, irinotecan, lurtotecan, exatecan or a pharmaceutically acceptable salt or metabolite thereof.

35. The kit of claim 31, wherein the topoisomerase inhibitor is topotecan or a pharmaceutically acceptable salt or metabolite thereof.

36. The kit of claim 31, wherein the cancer is selected from the group consisting of leukemia, prostate cancer, transitional cell carcinoma of the bladder, pancreatic cancer, colorectal cancer, cervical cancer, and lung cancer.

37. The kit of claim 31 further comprises an effective amount of a benzopyrone compound of formula (II): 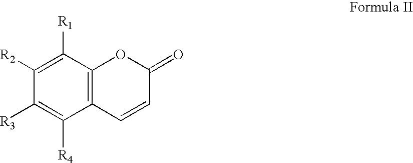
wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite or prodrug thereof.

38. The kit of claim 31 further comprises directions for administering the PARP inhibitor, the topoisomerase inhibitor or both.

39. The kit of claim 31, wherein the PARP inhibitor, the topoisomerase inhibitor, or both are in unit dosage form.
